(19) United States
(12) Patent Application Publication
(10) Pub. No.: US 2011/0201771 A1
(43) Pub. Date: Aug. 18, 2011

(54) BIOSYNTHESIS OF POLYISOPRENOIDS

(75) Inventors: Judit E. Puskas, Akron, OH (US); Colleen Mcmaham, Sausalito, CA (US); Alain M. Deffieux, Bordeaux (FR); Joseph P. Kennedy, Akron, OH (US)

(73) Assignees: UNIVERSITY OF AKRON, AKRON, OH (US); UNITED STATES OF AMERICA AS PRESENTED BY THE SECRE TARY OF AGRICULTURE, WASHINGTON, DC (US)

(21) Appl. No.: 12/998,516

(22) PCT Filed: Nov. 5, 2009
(86) PCT No.: PCT/US09/05985

§ 371 Date: Apr. 28, 2011

Related U.S. Application Data
(60) Provisional application No. 61/198,446, filed on Nov. 6, 2008.

(51) Int. Cl. C08F 36/08; C12P 5/02

(52) U.S. Cl. 526/193; 435/167

(57) ABSTRACT

The synthetic production of cis-1,4-polyisoprene and other cis-1,4-polydienes is achieved by adding isoprene or other diene monomers to a natural rubber latex or washed rubber particles and utilizing various allylic pyrophosphate compounds. The natural rubber latex or washed rubber particles inherently contain an enzyme catalyst and desirably divalent metal cofactors therein and the polymerization can occur over a relatively wide temperature range. The process is believed to be a living carbocationic polymerization. The in vitro produced natural rubber polymers can contain from about 5 to about 30,000 repeat units and are essentially free of non-enzyme catalysts. The invention can be utilized to synthesize polyisoprenoids and precursors to form terpenes, vitamins, steroids, alkaloids, and the like.

### CROSS REFERENCE

This patent application claims the benefit and priority of U.S. provisional application 61/198,446, filed Nov. 6, 2008 for BIOSYNTHESIS OF POLYISOPRENOIDS, which is hereby fully incorporated by reference.

This invention was made with government support under Grant No. NSF-CHE-0616834 awarded by the USDA. The Government has certain rights in the invention.

### FIELD OF THE INVENTION

The present invention relates to the synthetic, i.e. in vitro, production of natural rubber and other polyisoprenoids using synthetic monomers and initiators together with active enzyme catalysts. Natural rubber is produced utilizing active natural latex feed stock or washed rubber particles inherently containing an enzyme catalyst, and divalent metal cofactors necessary for enzyme activity. Other polyisoprenoids (steroids, vitamins etc) are produced with the appropriate natural active enzyme. More specifically, the natural isopentenyl pyrophosphate monomer is replaced by isoprene or other diene monomers, and the natural initiators may also be replaced with synthetic initiators containing end groups that are reminiscent of the allylic pyrophosphate end groups of naturally occurring initiators.

It is believed that the polymerization with IPP proceeds by a combination of chain growth polymerization and polycondensation, similarly to other biological polymerizations such as peptide elongation, and DNA and RNA biosynthesis. It is also believed that the polymerization proceeds by a mechanism that generally fits the definition of living polymerization established by the IUPAC (International Union of Pure and Applied Chemistry). This living-like character of the polymerization allows one to perform chain end functionalization (using functional allylic initiator or a functional terminating agent) as well as to prepare block copolymers, via a carbocationic mechanism. In the case of IP or other diene monomers the polymerization mechanism may be different since the monomer does not contain a pyrophosphate group.

The number average molecular weight of the polymers can vary over a wide range. In the case of using natural rubber latex or washed rubber particles the end product is a high purity cis-1,4-polyisoprene that is essentially free of non-enzyme catalysts, and has substantially reduced protein content.

### BACKGROUND OF THE INVENTION

### Natural Rubber Biosynthesis—A Living Carbocationic Polymerization

### 1. Introduction

Let us set the stage by quoting de la Torre and Sierra [1]: “The appealing beauty of the routes that Nature uses to build natural products is breathtaking and the quest for laboratory syntheses that mimic these routes is longstanding”. The exponential rise in the syntheses of “bio-inspired” polymers, and the use of enzymes to mediate organic reactions powerfully underlines this view [2-9]. Enzymatic catalysis in vitro, i.e., under simulated physiological conditions, leads to products identical to “natural” (in vivo) compounds, or to an “artificial” version of the natural product [10-12]. Bacterial enzymes have been used to synthesize polyesters in vitro [13-18], and a variety of isolated enzymes have been used both in vitro and under “artificial” conditions [15, 19-40]. Further recent examples are the syntheses of functionalized amphiphilic polymers and polyesters, and the ring-opening polymerization of seven-membered ring lactones catalyzed by immobilized Candida Antarctica [41-43]. Enzymatic catalysis has also been used to initiate polymerizations from surfaces [44-47].

The selectivity, efficiency and non-toxicity of enzymes render them attractive catalysts. The study of natural and biomimetic organic syntheses may also yield non-enzymatic processes, and novel “synthetic methodology inspired by biogenesis” [1, 48-52]. Yokozawa et al.'s [48,49] recent work is particularly revealing in this context. Inspired by the biosynthesis of many natural biopolymers, these authors developed the concept of “chain-growth polycondensation”, according to which an enzyme activates the initiating entity and/or the dormant polymer chain end, which proceeds to add monomer and relinquishes the protective end group. FIG. 1 helps to visualize this concept. Examples cited by these authors include peptide extension (termed “elongation” in biochemistry) [53,54], DNA [55] and RNA [56] syntheses and natural rubber biosynthesis [57-59]. Yokozawa's group developed two strategies for chain-growth polycondensation. The first strategy involved the activation of polymer end groups by substituents, and led to aromatic polyamides, polyesters, polyethers, poly(ether sulfone) and polythiophene. By the second strategy the monomer was separated from the polymerization phase to prevent monomer-monomer and polymer-polymer condensations. Yokozawa's work, while a breakthrough in bio-mimetic polymer synthesis, did not produce the desired monodisperse polymers, such as specific polypeptides or DNA. The synthesis of cPIP was attempted by the use of an electrophilic initiator by the pathway shown in FIG. 2, however, only a diene was obtained [48]. The proposed chain-growth polycondensation mechanism should lead to monodisperse cPIP; however, all natural rubbers exhibit multimodal/broad molecular weight distributions [60,61].

### 2. Natural Rubber

2.1. Background

Natural rubber, arguably the most important polymer produced by plants, is a strategically important raw material used in many thousands of products, including hundreds of medical devices. Natural rubber is obtained from latex, an aqueous emulsion present in the laticiferous vessels (ducts) or parenchymal (single) cells of rubber-producing plants. Although more than 2,500 plant species are known to produce natural rubber, currently there is only one important commercial source, Hevea brasiliensis (the Brazilian rubber tree). The rubber from Parthenium argentatum, also called guayule, is being marketed as “non-allergenic natural rubber” [60-63]. We do not know why plants produce rubber and our understanding of the mechanism of natural rubber biosynthesis is far from complete.

The rubber latex from H. brasiliensis is harvested by “tapping” the rubber tree, i.e., making an incision in the trunk and collecting the sap freely oozing out of the ducts. The raw polymer is recovered from the latex by coagulation and drying, yielding high molecular weight (>1 million g/mol) “crepe”. While Charles Goodyear is credited with discovering the crosslinking of natural rubber by sulfur in 1839, ancient Mesoamerican peoples discovered the advantages of crosslinking much earlier: they mixed the rubber latex harvested from the Castilla elastica tree with the juice of Ipomoea alba (a species of morning glory vine) and produced solid rubber. Recent analysis of Olmec rubber balls (1600-1200 B.C.) could not identify the exact chemical nature of the crosslinks, although the dynamic mechanical properties of the rubber crosslinked by the ancient method were found to resemble closely those of modern vulcanized NR [64]. Vulcanized natural rubber exhibits an excellent combination of properties, including elasticity, resilience, abrasion resistance, efficient heat dispersion and impact resistance, which led to sustained research and development efforts to develop synthetic processes to produce natural rubber. To date, this objective has not been achieved.

Despite extensive research, the exact structure of natural rubber is still unknown. Early X-ray diffraction studies showed that the double bonds of the isoprene repeat units are in cis configuration [65]. Later Tanaka et al. [66], by the use of ¹H-NMR and ¹³C-NMR spectroscopy, showed that the second and third units of Hevea rubber are trans, followed by repetitive cis enchainment (FIG. 3). The terminal groups are believed to be —CH₂OH (or a fatty acid ester). The presence of cyclized polyisoprene sequences in NR was also detected. Other chemical groups, termed “abnormal” were also identified (aldehydes, epoxides and amines) however, their origin remains unknown. H. brasiliensis and P. argentatum produce high molecular weight (Mₙ=10⁵−10⁶ g/mol) and broad/bimodal molecular weight distribution (M_{w}/Mₙ=2−10) NR [60, 61, 67-69]. Both rubbers contain gelled (crosslinked) fractions, but it is not known if gelation occurs in the plants or arises during processing. The chemical structure of natural cPIP rubbers obtained from different plant species (Russian dandelion, goldenrod, Jelutong, etc.) differs only in the number of the initial trans units (0 to 3) following the 1,1-dimethylallyl head group. Only a relatively few species, such as balata and Gutta percha, produce gutta or trans-1,4-PIP. Chicle produces a ¼ mixture of cPIP and trPIP [60,61].

It is well-established that solid natural rubber from various sources can often contain from about 50 to about 70 wt. % water [70] and are generally stabilized by a membrane of a phospholipid monolayer (FIG. 4) [71] and contain a cis-prenyltransferase enzyme that is not yet fully characterized. [72,73]. Natural rubbers such as Hevea brasiliensis are composed of long-chain branched molecules. [74,75] Fulton et al. showed the presence of long-chain branching in NR by Field Flow Fractionation (FFF): the log R_{g}-log MW conformational plot was shown to have a slope of 0.3 in the high MW range. [76] NR also contains 50-70% gel produced by hydrogen bonding between proteins, the so-called “soft” gel, and by phospholipid-based crosslinking points, which form the so called “hard” gel. It has been shown that the addition of 1-2% ethanol into a solution with a good solvent, such as toluene, dramatically decreases the gel content in NR by breaking the hydrogen bonds that make up the “soft” gel, as shown in FIG. 5. [73,77] The phospholipid-based crosslinking points can be broken via transesterification. [78,79].

At the present no synthetic cPIP is able to mimic the performance of natural rubber [80, 81]. Thus natural rubber is an essential renewable resource material. Very high cis-content (more than 99,99% 1,4 cis polyIP) is claimed in the literature [82, 83, 84, 85]; 97% PIP is produced by titanium-based Ziegler-Natta catalysts [86], and anionic polymerization produces up to 96% cPIP with the rest in 3,4-vinyl enchainment [87, 88]. The so-called “low-cis” anionic PIP still contains ˜92% cis-enchainment [89], however, the properties of this product are inferior to high-cis PIPs produced by Ziegler-Natta catalysts. High cPIP can also be obtained by the use of neodymium-based catalysts [90]. Metallocene-catalyzed isoprene polymerizations are also mentioned in the literature, although dienes are believed to be poisons for these catalysts [81]. FIG. 6 shows the different microstructures obtained by the various techniques.

According to our extensive literature survey, 100% cPIP has never been produced synthetically. The outstanding overall properties of NR are mainly attributed to the 100% cis microstructure, together with specific molecular weights and molecular weight distributions of the cPIP, and the presence of proteins and the so-called “abnormal” groups [60, 61, 66]. Thus the synthesis of NR-like 100% cPIP would be of considerable fundamental and practical significance.

The next section concerns a brief overview of NR biosynthesis.

2.2. The Biosynthesis of Natural Rubber

2.2.1. Biosynthesis In Vivo

The biosynthesis of NR is catalyzed by the rubber transferase (cis-prenyl transferase [66, 91-96]) enzyme(s), bound to rubber particles in the latex. Polymerization takes place within the boundary phospholipid biomembrane monolayer, located between the nonpolar rubber particles and the aqueous medium [97]. The hydrophobic chains are sequestered inside the latex particles, and the phospholipids monolayer stabilizes the particles preventing aggregation.

The polymerization proceeds at the active sites of the amphiphilic enzyme [71, 98], which contains glycosylated hydrophilic regions that mediate the access of incoming hydrophilic building blocks and hydrophobic regions that mediate their placement into the biomembrane [99]. The monomer, isopentenyl pyrophosphate IPP, generated from carbohydrates in plants, is associated in vivo with monovalent cations (Mt⁺: K⁺, Na⁺ or NH₄⁺) [68, 69, 100, 101]. FIG. 7 shows the structure of IPP, chemically an adduct of pyrophosphoric acid (H₄P₂O₇) and isoprene (IP). IPP is isomerized to 1,1-dimethylallyl pyrophosphate (DMAPP) by IPP isomerase (FIG. 7). Catalyzed by specific trans-prenyl transferases [92], DMAPP adds 1-3 IPP units to form oligomeric allylic pyrophosphates (APPs, FIG. 8), all of which may function as initiators.

The IPP and the APPs are termed “substrate” and “cosubstrates”, respectively, in the biochemical literature. These entities would be termed monomer and initiator by polymer chemical terminology. Enzymatic activity requires the presence of divalent cations, such as Mg²⁺ or Mn²⁺, called “activity cofactors” [91-94, 102]. The substrate and cofactors are hydrophilic, the cosubstrates are either hydrophilic or amphiphilic, while the rubber product is hydrophobic. The amphiphilic enzyme is located at the interface between the rubber particles and the aqueous phase of the latex. FIG. 9 shows a flow diagram of NR biosynthesis, reproduced from the biochemical literature [69]. FIG. 10 shows the biosynthesis of NR in terms of the initiation and propagation steps proposed by Tanaka [66], i.e., in polymer chemical symbolism. In each step, one molecule of pyrophosphoric acid (HPP or its salts) is generated, i.e., the process is a combination of chain growth and polycondensation, as suggested by Yokozawa [47,48]. Molecular weights and molecular weight distributions are species-dependent; however, the biochemical literature is mute in regard to the control of these parameters in vivo [69]. According to the polymer chemical literature, broad/multimodal molecular weight distributions are due to branching and/or crosslinking by acid-catalyzed cyclization, or by “abnormal” functional groups (aldehydes, epoxides and amines) [60, 61, 66].

2.2.2. Biosynthesis In Vitro

The in vitro biosynthesis of NR has been demonstrated [68, 69, 100], however, only at the mg scale due to the extremely limited availability of reagents. The elucidation of NR biosynthesis in terms of polymer chemical principles may lead to a synthetic strategy for 100% cPIP. Our exhaustive literature search revealed surprisingly little information in regard to polymer chemical aspects of NR biosynthesis, and we found no evidence for ongoing research in this area by the contemporary polymer chemical community. In contrast, the biochemical community is quite active in this area. It is argued that global dependence on one species, H. brasiliensis, as a single source of NR is risky (guayule production is still quite limited), and that current H. brasiliensis crops have very little genetic variability, leaving rubber plantations at risk of serious pathogenic attacks. In addition, repeated exposure to residual proteins in latex products derived from H. brasiliensis have led to serious and widespread allergenic (Type I) hypersensitivity [103-107]. Thus, alternative sources of NR are eagerly sought by the biochemical community [108].

Archer and Audley were first to initiate the in vitro synthesis of cPIP by neryl pyrophosphate (NPP, C₁₀), a cis-allylic pyrophosphate [100]. They incubated ¹⁴C-IPP in the presence of unlabelled neryl or geranyl pyrophosphate initiators in a suspension of washed rubber particles (isolated from living H. brasiliensis latex), and demonstrated that the cis-allylic neryl pyrophosphate was a more efficient initiator than the trans-allylic geranyl pyrophosphate. The same authors found that the rate of IPP incorporation increased with the chain length of the APP oligomer (DMAPP<GPP<FPP<GGPP). Cornish et al. found the same trend for P. argentatum [96]. Cornish's team [69] developed three in vitro NR synthesis systems (H. brasiliensis, P. argentatum and Ficus elastica), and concluded that rubber transferases are not particularly sensitive to the size and stereochemistry of the initiator. Similarly to in vivo NR biosynthesis, the molecular weight and the molecular weight distribution of the rubber depended on the species from which the living latex was harvested. This group also showed that increasing the FPP concentration increased the IPP incorporation rate, but decreased the molecular weight; and increasing the IPP concentration increased both initiation and propagation rates, as well as molecular weights [68, 96]. Based on their results these workers concluded that the detachment of the rubber molecule from the rubber transferase is not the primary regulator of molecular weights in vivo. These pioneering studies generated valuable insight into the mechanism of NR biosynthesis.

It has been known since the 1950s that the chain elongation of rubber molecules proceeds by the addition of the isopentenyl pyrophosphate (IPP) to polyisoprenoids. [109,110] Based on the mechanism of low molecular weight terpenoid biosynthesis, the initiator was assumed to be dimethylallyl pyrophosphate (DMADP). [111] However, the addition of radioactive ¹⁴C DMADP into fresh Hevea latex did not form new rubber chains containing the radioactive head group. [73] In line with this, Tanaka did not find the dimethyl allyl head group in Hevea rubber, and concluded that the currently accepted initiation mechanism, shown in FIG. 10, remains unproven. [71-73] It has also been postulated that the alignment of trans-isoprene units is derived from the initiating species such as trans,trans-farnesyl diphosphate (FDP) (with DP=PP) or trans, trans, trans-geranylgeranyl diphosphate (GGDP). The chain elongation step of rubber formation may start from FDP or GGDP by successive condensation of IDP to form a long sequence of cis-isoprene units. [112]

To this date, genetic sequences of the rubber transferase(s) remain unidentified because it is a membrane-bound enzyme in low abundance. [71] Conventional continuous assays to determine the enzymatic activities are a challenge due to the fact that the activity of the rubber particles is rapidly lost upon disruption of their structural integrity [113]. The present method to determine the activity of rubber transferases is by radiometric assay where the activity is calculated based on the incorporation rate of ¹⁴C IPP monomer into higher molecular weight rubber produced in vitro. [113,114].

Benedict et al. produced in vitro guayule rubber using WRP, synthetic IPP as monomer and DMAPP as initiator in a reconstituted latex. [115] Their WRP was prepared from stems of P. argentatum; after removal of the bark and impurities, the rubber latex was centrifuged and the top rubber particulate layer was collected and purified by repeated washing with buffer and centrifugation as the WRP. The authors used a SEC coupled with a scintillation spectrometer to demonstrate that radioactive IPP became incorporated into NR. [115] They observed that rubber was formed with a peak molecular weight of ˜10⁵ g/mol within 15 minutes, and that the rubber was able to grow to ˜10⁶ g/mol in three hours [115].

Tanaka's group established a novel method for in vitro rubber biosynthesis using the fresh bottom fraction (BF) of the latex. [116,117] They employed a muslin cloth to filter out some of the coagulants and then centrifuged the liquid latex. The number of washing cycles applied to the latex influenced the amount of rubber produced. The washed latex was partitioned into three major fractions: the top particulate layer, a middle clear phase, which was called C-serum (CS), and a bottom fraction marked BF. [116, 117] The BF was used for in vitro NR biosynthesis and it was observed that more than ˜10 wt % new rubber formed with the addition of very small amounts of IPP or FPP to fresh BF. [118] It was also found that new rubber formed by the incubation of BF without the addition of IPP or FPP, concluding that the BF contains all the enzymes and precursors necessary to produce rubber. [139] The formation of new rubber was confirmed by the incorporation of ¹⁴C labeled IPP into the resulting rubber. [116] FIG. 11 compares the UV traces of the endogenous NR from BF and the in vitro NR rubber. The BF has a high molecular weight fraction around ˜10⁶ g/mol, with a lower MW tail. The newly formed rubber produced a peak at about ˜10⁵ g/mol. The radioactive traces also revealed that while the ¹⁴C-IPP incorporated into new chains, it also added to pre-existing chains in the lower MW tail fraction of the BF. [116]

Wititsuwannakul's group in Thailand also developed their unique Washed Bottom Fraction Particles WBP for in vitro NR biosynthesis. [119,120] This system also incorporated radioactive IPP into NR. More recently, this group cloned two suspected gene sequences of Hevea rubber transferase and expressed them in E. Coli. [72] It was found that a combination of one of the clones with WBP resulted in enhanced NR growth.

Cornish et al. developed the method to produce WRP that is utilized in our studies. This method is an improvement over that of Benedict's, in that the rubber particles from the top fraction are collected and purified by repeated washes with buffer, centrifugation, and re-suspension of the top fraction in a buffer. [93] Her group determined the MW of in vitro NR by means of dual-labeled liquid scintillation spectrometry (SS). [69] By introducing both radioactive IPP monomer and FPP initiator into in vitro NR biosynthesis and calculating the incorporation rate of the radioactive materials, an average MW of the newly formed rubber was calculated from the ratio of ¹⁴C-labeled monomer to ³H-labeled initiator. [69] It is important to note that the proposed MW calculation assumes that chain growth starts only from the synthetic FPP initiator and the IPP monomer does not add to pre-existing rubber. [69] This assumption contradicts Tanaka's results mentioned above. The reported MWs were in the 10⁴-10⁵ g/mol range, [69,96] somewhat lower than those reported by Tanaka's and Wititsuwannakul's group.

Strikingly, the exact chemical macro- and microstructure of NR remain unknown; the current understanding of NR structure is based upon naturally occurring model compounds. [121]

### 3. The Mechanism of Natural Rubber Biosynthesis: A Living Carbocationic Polymerization

In this section we assemble and analyze information pertaining to the biosynthesis of NR in the biochemical literature, and translate it into polymer chemical formalism, i.e., initiation, propagation and termination. While some of the basic steps of NR biosynthesis have been elucidated from the biochemical point of view, our understanding of this process in terms of synthetic polymer chemistry is practically nonexistent.

FIG. 12 shows Archer et al.'s mechanism [109] of initiation in the enzymatic synthesis of cPIP. According to this mechanism, first an enzyme (En) interacts with the phosphorylated monomer IPP to form an En-IPP adduct, which reacts with another IPP while HPP is lost. The product is able to repeat (and sustain) IPP addition with the simultaneous loss of HPP. An allylic pyrophosphate initiator is not required. Originally, this mechanism was proposed as an alternative to McMullen's theory [122], according to which propagation occurs by IPP-nucleotide complexes, and the nucleic acid double helix is a template for stereospecific polymerization [122]. Consideration of biological precedents led to the development of templated polymerizations proceeding with stereochemical restrictions, and supramolecular assembly [123-128].

Neither McMullen nor Archer et al. addressed the role of the divalent cation cofactors, and both propositions remain unsubstantiated. While the exact role of the cofactors is still unclear, Scott et al. [102] recently demonstrated that only cofactor-activated IPP monomer will interact with the enzyme, while the FPP initiator may bind even in the absence of cofactors. Two Hevea cis-prenyltransferase cDNAs have recently been sequenced [72], but the chemistry of natural rubber biosynthesis is still incompletely understood.

We wish to present a new view of NR biosynthesis from the point of view of synthetic polymer chemistry. Recent insight into the mechanism and kinetics of living carbocationic polymerization [129] was essential to develop this proposal.

In regard to polymer chemical terminology, the various allylic pyrophosphates are initiators, the IPP is the monomer, and the rubber transferase in association with the divalent cation co-factors is the coinitiator. Thus the elementary steps of NR biosynthesis can be described in terms of initiation, propagation and termination.

3.1. Initiation

A close inspection of NR biosynthesis leads us to postulate that the structures of the intermediates involved in this process are consistent with a carbocationic polymerization mechanism. FIG. 13 shows the proposed mechanism for geranyl pyrophosphate (GPP=trans,trans) initiator, however, the same process could also be formulated with neryl (NPP=cis,cis) pyrophosphate, etc. as well as other APPs.

Initiation starts by an enzyme (and divalent cationic cofactor)—assisted ionization of the carbon-oxygen bond of the initiator (GPP, etc.) and yields an allylic cation plus pyrophosphate counteranion; the enzyme plus cofactor(s) coordinates with the pyrophosphate “protecting” group and mediates the formation of the initiating carbocation. According to polymer chemical convention, the enzyme plus cofactors constitute the coinitiating system (catalytic system) (FIG. 14). Ionization at the chain end is favored by resonance stabilization of the allylic carbocation and increasing entropy of the system. This mechanism is unlikely with the IPP monomer because the cleavage of the carbon-oxygen bond would lead to an energetically unfavorable primary carbocation (FIG. 7). Subsequently, the vinylidene group of IPP adds to the allylic carbocation, yielding a tertiary carbocation which, via proton elimination, regenerates the trisubstituted allylic pyrophosphate. This mechanism applies to the formation of trans-1,1-dimethylallylic initiators (FIG. 8), catalyzed by trans-prenyl transferase, as well as to the incorporation of the first cis-unit, i.e., initiation, catalyzed by cis-prenyl transferase (FIG. 10). In regard to trans or cis stereoregulation, the specific enzyme functions as the template, and yields exclusively trans-1,4 or cis-1,4 incorporation—this will be explained in more detail later. The incorporation of each IPP unit is always accompanied by the loss of pyrophosphoric acid HPP (or its salts).

3.2. Propagation

Propagation proceeds by the same cationation/IPP-addition/proton loss sequence as occurs in initiation (FIG. 10). The initiation and propagation steps are fundamentally similar to those in living carbocationic (and living radical) polymerization governed by dormant-active equilibria [130, 131]. The difference is that in the natural process every propagation step is followed by deactivation, accompanied by the release of HPP, while in synthetic living carbocationic or radical polymerization the active chain end adds more than one monomer units before temporary deactivation [129, 130].

Poulter et al.'s work on prenyl transfer reactions in natural terpenoid synthesis supports the possible involvement of carbocationic species in NR biosynthesis [111, 132-134]. According to these authors [132] resonance-stabilized allylic carbocationic intermediates arise during the reaction of allylic pyrophosphate with IPP (FIG. 15). The dissociative (S_{N}1) mechanism was substantiated by an excellent Hammet plot for rates of solvolysis versus prenyl transfer with fluorine substituted APP. Enzyme-catalyzed hydrolysis of GPP in H₂¹⁸O revealed breakage of the C—O bond and inversion of chirality at the C1 carbon [134]. This mechanism is generally accepted by the biochemical community [135].

Polymer chemists, who may be skeptical in regard to a carbocationic polymerization proceeding in an aqueous medium, should recall that carbocations can be generated in aqueous media under select conditions. Thus, Sawamoto et al. described numerous cationic polymerizations in aqueous systems [136, 137], and Mayr et al. demonstrated that carbocations can react with π nucleophiles (e.g., olefins) in aqueous media under appropriate conditions [138].

A unique feature of in vivo NR biosynthesis is the control of proton loss from the tertiary carbocation yielding exclusively 1,4-enchainment (III in FIG. 16). According to classical organic chemistry proton loss from a tertiary carbocation leads to isomer mixtures I, II and III (FIG. 16). The exclusive formation of III in vivo suggests a more acidic proton between the tertiary carbocation and the —CH₂—PP group, or a concerted mechanism involving one of the pyrophosphate groups and/or the enzyme. Hevea rubber biosynthesis in vivo also yields exclusively cisIII, but as mentioned above, some plants produce transIII, or a mixture of cis and transIII.

The proposed carbocationic mechanism is consistent with the observed broad molecular weight distributions and cyclized sequences present in NR; new chains are continuously initiated, while intermolecular attack of the growing carbocation on a double bond of another polymer chain may lead to branching and broad/multimodal distributions, and intramolecular attack to cyclization. Fresh latex was shown to exhibit monomodal molecular weight distribution [60, 61], while processed Hevea rubber displays multimodal distribution. These facts can be readily explained by chain-chain coupling under acidic conditions. Chain-chain coupling and cyclization have been observed in the carbocationic polymerization of isoprene [139, 140]. Terpenoid cyclization occurring in plants is also believed to proceed by carbocationic mechanism [135]. It was suggested that the “abnormal” groups arise during processing [60, 61]. In our view, only initiation, propagation and temporary deactivation proceed in vivo. Cis-trans stereoregulation may be due to specific enzymes acting as templates for monomer incorporation.

Evidence for this view came from an analysis of NR biosynthesis (see above), combined with an evaluation of recent progress in sequencing and 3D structure determination of various prenyltransferases enzymes, as well as other biochemical studies concerning the mechanisms of isoprenoid syntheses [135]. For example, in prenyl transfer reactions the binding sites for the initiating 1,1-dimethylallylic pyrophosphates and IPP were located within the hydrophilic regions of the enzyme, whereas chain growth was proposed to take place within a hydrophobic pocket positioned toward the bottom end of the conical enzyme (FIG. 17).

According to a polymer chemist view, and in agreement with the literature on NR biosynthesis, the amphiphilic enzyme resides at the interface (FIG. 4). Propagation occurs as suggested [135], when the allylic end of the initiator or the NR molecule is docked at the specific site within the tunnel-like crevice of the enzyme and is activated, and IPP assisted by divalent cofactors enters from the aqueous phase. Stereoregulation within the interior of the enzyme is visualized by steric restriction—the spatial arrangement and size of the various microstructures shown in FIG. 6 are compatible with a specific enzyme pocket. The rubber molecule may be released from the docking site after the incorporation of IPP, and a new rubber molecule may be docked and activated. We propose that the rubber molecules compete for the active sites; this alone can lead to broad molecular weight distributions. Continuous supply of IPP monomer and APP initiator to the particle will also lead to broad distribution. Chain growth stops when the rubber chain reaches a critical molecular weight, which prevents further diffusion of large molecules to the interface. The critical molecular weight is species-dependent, and is most likely based on the stabilization and composition of the latex. Permanent termination does not occur in vivo, only during processing.

3.3. Termination

Termination in isoprenoid biosynthesis was proposed to be due to the position of specific large motifs (see FIG. 17) that stop chain growth at a specific length [135]. This is an attractive suggestion for the formation of short isoprenoids, but not for Hevea rubber that has ˜5000 repeat units. With increasing chain length the NR molecule becomes increasingly hydrophobic and will extend beyond the enzyme pocket, thus chain length regulation by this mechanism is inconceivable. According to the biochemical literature pertinent to NR biosynthesis, chain termination occurs when the enzyme relinquishes the rubber molecule. If the detachment of the enzyme (termination) were to occur at a specific chain length regulated by steric factors as suggested for short-chain isoprenoids, uniform NR would arise. Indeed, such short-chain isoprenoids with well-defined chain lengths (up to 24-mers) arise in plants catalyzed by various cis- and trans-prenyltransferases [135]. However, NR exhibits broad molecular weight distribution. The proposed chain activation involving the enzyme and cofactors (see FIGS. 13 and 14) implies that the enzyme is released after each propagation step. Evidently, the polymerization rate is governed by the rate of IPP addition relative to that of activation of the dormant rubber molecule. Random activation and migration/docking of the rubber molecule to the activating site of the enzyme lead to broad molecular weight distributions.

The NR molecule contains HO-end groups, which most likely arise by hydrolytic cleavage of terminal polymer-pyrophosphate linkages (FIG. 18). The chain end may also react with fatty acids, which leads to ester end groups. It is unknown whether hydrolytic termination occurs in vivo or during processing. At physiological pH, the pyrophosphate end group is a quite stable dianion [112]. Post-polymerization reactions have been described [60, 61]. We believe that termination by hydrolysis does not occur in vivo, because this would prevent the formation of high molecular weight rubber. Species-specific molecular weight regulation is conceivably due to physical factors, such as the size of the latex particle and/or stabilization by specific proteins in the plant.

### 4. Conclusion

### Natural Living Carbocationic Polymerization (NLCP)

Based on the above comprehensive review and analysis of the chemical, polymer chemical and biochemical literature pertaining to the biosynthesis of NR, we developed a new mechanistic view of this process. First, we wish to stress that this biosynthesis proceeds by a combination of chain growth polymerization and polycondensation, similarly to other biological polymerizations such as peptide elongation, and DNA and RNA biosynthesis. It is also proposed that the polymerization proceeds by a mechanism that fits the definition of living polymerization set forth by the IUPAC: “Living polymerization is a chain polymerization from which chain transfer and chain termination are absent.” Second, all the species identified by earlier authors that are known to arise during biosynthesis can be described in terms of carbocationic intermediates. A combination of these two critical parameters, livingness and carbocationic intermediates, leads us to propose that the biosynthesis of NR proceeds by a natural living carbocationic polymerization mechanism (NLCP). FIG. 19 summarizes the concept of NLCP.

The polymerization starts by initiation, which involves two events: ionization or priming (tantamount to activation in biochemical parlance) and cationation. During ionization by an activator (Y) the allylic end group of an initiator (see FIG. 19) yields a resonance stabilized primary/tertiary allylic cation. In NR biosynthesis ionization is mediated by specific transferases and assisted by inorganic cofactors (see FIG. 13), while the NLCP concept is inclusive of other possible activators which produce the desired resonance-stabilized allylic cation. If the activated chain end cationates incoming monomer (MX), a tertiary carbocation intermediate (shown in brackets) is formed which promptly loses a proton (i.e., HX is lost). MX is a “protected” monomer which cannot be ionized by the activator Y. The counteranion X⁻ is a pyrophosphate residue in plants, however in NLCP it may be a different anion. Propagation is repetitive ionization/cationation: the allylic terminus is ionized (activated) by Y, reacts with the incoming monomer, and the intermediate tertiary carbocation, via HX loss, regenerates exclusively the allylic chain end. This sequence of events sustains propagation by the same mechanism that prevails in initiation. Thus NLCP can be viewed as a controlled/living carbocationic polymerization with dynamic equilibria between dormant and active species, combined with controlled loss of HX (importantly, HX loss after each monomer incorporation must regenerate exclusively the dormant allylic chain end—III in FIG. 19).

Pₙ—X+Y [symbol] Pₙ⁺//⁻X—Y,

Pₙ⁺//⁻X—Y+MX→Pₙ₊₁⁺X//⁻X—Y→Pₙ₊₁—X+HX+Y

The NLCP mechanism, combined with cis-trans stereoregulation (for example, by the activator or by a suitable template), may serve as a blueprint for the design of synthetic systems emulating the biosynthesis of NR, and of many other polyterpenes.

### REFERENCES

[1] De la Torre M C, Sierra M A. Comments on Recent Achievements in Biomimetic Organic Synthesis. Angew. Chem. Int. Ed 2004; 43:160-181.
[2] Sandford K, Kumar M. New Proteins in a Materials World. Curr. Op. in Biotechnol. 2005; 16:416-421.
[3] Cunliffe D, Pennadam S, Alexander C. Synthetic and Biological Polymers—Merging the Interface. Eur. Polym. J. 2004; 40:5-25.
[4] Aizenberg J, Landis W J, Orme C, Wang R eds. Biological and Bioinspired Materials and Devices. Mat. Res. Soc. Symp. Proc. 2004; 823.
[5] Kobayashi S, Uyama H. Enzymatic Polymerization to Polyesters. Biopolymers 2002; 3a:373-400.
[6] Kobayashi S, Uyama H, Kimura S. Enzymatic Polymerization. Chem. Rev. 2001; 101:3793-3818.
[7] Kobayashi S. Enzymatic Polymerization: A New Method of Polymer Synthesis. J. Polym. Sci., Part A: Polym. Chem. 1999; 37:3041-3056.
[8] Uyama H, Kobayashi S. Enzymatic Polymerization Yields Useful Polyphenols. Chemtech. 1999; 29:22-28.
[9] Barron A E, Zuckermann R N. Bioinspired Polymeric Materials: In-Between Proteins and Plastics. Curr. Opin. Chem. Biol. 1999; 3:681-687.
[10] Kobayashi S, Morii H, Ito R, Ohmae M. Enzymatic Polymerization to Artificial Hyaluronic Acid Using a Transition State Analogue Monomer. Macromol. Symp. 2002; 183:127-132.
[11] Kobayashi S, Uyama H. In Vitro Biosynthesis of Polyesters. Adv. Biochem. Eng/Biotechnol. 2001; 71:241-262.
[12] Burzio L A, Burzio V A, Pardo J, Burzio L O. In Vitro Polymerization of Mussel Polyphenolic Proteins Catalyzed by Mushroom Tyrosinase. Comp. Biochem. Physiol., Part B: Biochem. Mol. Biol. 2000; 126:383-389.
[13] Kamachi M, Zhang S, Goodwin S, Lenz R W. Enzymatic Polymerization and Characterization of New Poly(3-hydroxyalkanoate)s by a Bacterial Polymerase. Macromolecules 2001; 34:6889-6894.
[14] Su L, Lenz R W, Takagi Y, Zhang S, Goodwin S, Zhong L, Martin D P. Enzymatic Polymerization of (R)-3-Hydroxyalkanoates by a Bacterial Polymerase. Macromolecules 2000; 33:229-231.
[15] Broom A D, Uchic M E, Uchic J T. Combined Enzymatic and Chemical Approaches to the Synthesis of Unique Polyribonucleotides. Biochim. Biophys. Acta. 1976; 425:278-286.
[16] Livingston D C, Dale R M, Ward D C. The Synthesis and Enzymatic Polymerization of 5-thio- and 5-methylmercurithio-pyrimidine Nucleotides. Biochim. Biophys. Acta. 1976; 454:9-20.
[17] Scheit K H. Enzymatic Polymerization of S4-methyl-4-thiouridine-5′-diphosphate by Polynucleotide Phosphorylase from Escherichia coli. Biochim. Biophys. Acta. 1970; 209:445-454.
[18] Meadow P M, Anderson J S, Strominger J L. Enzymatic Polymerization of UDP-acetylmuramyl.L-ala.D-glu.L-lys.D-ala.D-ala and UDP-acetylglucosamine by a Particulate Enzyme from Staphylococcus aureus and its Inhibition by Antibiotics. Biochem. Biophys. Res. Commun. 1964; 14:382-387.
[19] Metral G, Wentland J, Thomann Y, Tiller J C. Biodegradable Poly(ester hydrazide)s Via Enzymatic Polymerization. Macromol. Rapid Commun. 2005; 26:1330-1335.
[20] Ochiai H, Ohmae M, Mori T, Kobayashi S. Bottom-Up Synthesis of Hyaluronan and Its Derivatives via Enzymatic Polymerization: Direct Incorporation of an Amido Functional Group. Biomacromolecules 2005; 6:1068-1084.
[21] Nakamura I, Yoneda H, Maeda T, Makino A, Ohmae M, Sugiyama J, Ueda M, Kobayashi S, Kimura S. Enzymatic Polymerization Behavior Using Cellulose-Binding Domain Deficient Endoglucanase II. Macromol. Biosci. 2005; 5:623-628.
[22] Kobayashi S, Itoh R, Morii H, Fujikawa S-I, Kimura S, Ohmae M. Synthesis of Glycosaminoglycans via Enzymatic Polymerization. J. Polym. Sci., Part A: Polym. Chem. 2003; 41:3541-3548.
[23] Kurisawa M, Chung J E, Kim Y J, Uyama H, Kobayashi S. Amplification of Antioxidant Activity and Xanthine Oxidase Inhibition of Catechin by Enzymatic Polymerization. Biomacromolecules 2003; 4:469-471.
[24] Hu Y, Ju L-K. Lipase-Mediated Deacetylation and Oligomerization of Lactonic Sophorolipids. Biotechnol. Prog. 2003; 19:303-311.
[25] Taden A, Antonietti M, Landfester K. Enzymatic Polymerization towards Biodegradable Polyester Nanoparticles. Macromol. Rapid. Commun. 2003; 24:512-516.
[26] Robert J P, Uyama H, Kobayashi S, Jordan R, Nuyken O. First Diazosulfonate Homopolymer by Enzymatic Polymerization. Macromol. Rapid Commun. 2003; 24:185-189.
[27] Fukuoka T, Tachibana Y, Tonami H, Uyama H, Kobayashi S. Enzymatic Polymerization of Tyrosine Derivatives. Peroxidase- and Protease-Catalyzed Synthesis of Poly(tyrosine)s With Different Structures. Biomacromolecules 2002; 3:768-774.
[28] Kobayashi S, Morii H, Itoh R, Kimura S, Ohmae M. Enzymatic Polymerization to Artificial Hyaluronan: A Novel Method to Synthesize a Glycosaminoglycan Using a Transition State Analogue Monomer. J. Am. Chem. Soc. 2001; 123:11825-11826.
[29] Uyama H, Ikeda R, Yaguchi S, Kobayashi S. Enzymatic Polymerization of Natural Phenol Derivatives and Enzymatic Synthesis of Polyesters from Vinyl Esters. ACS Symp. Ser. 2000; 764:113-127.
[30] Shan J, Cao S. Enzymatic Polymerization of Aniline and Phenol Derivatives Catalyzed by Horseradish Peroxidase in Dioxane. Polym. Adv. Technol. 2000; 11:288-293.
[31] Sakamoto J, Sugiyama J, Kimura S, Imai T, Itoh T, Watanabe T, Kobayashi S. Artificial Chitin Spherulites Composed of Single Crystalline Ribbons of α-Chitin via Enzymatic Polymerization. Macromolecules 2000; 33:4155-4160.
[32] Park O J, Kim D Y, Dordick J S. Enzyme-Catalyzed Synthesis of Sugar-Containing Monomers and Linear Polymers. Biotechnol. Bioeng. 2000; 70:208-216.
[33] Kobayashi S, Shoda S I. Chemical Synthesis of Cellulose and Cello-Oligomers Using a Hydrolysis Enzyme as a Catalyst. Int. J. Biol. Macromol. 1995; 17:373-379.
[34] Aizawa M, Wang L L, Shinohara H, Ikariyama Y. Enzymatic Synthesis of Polyaniline Film Using a Copper-Containing Oxidoreductase: Bilirubin Oxidase. J. Biotechnol. 1990; 14:301-309.
[35] Blandin M, Drocourt J L. Enzymatic Polymerization of 5-mercuriuridine-5′-diphosphate with Polynucleotide Phosphorylase from E. coli. Biochimie. 1984; 66:645-650.
[36] Sagi J T, Szabolcs A, Szemzo A, Otvos L. Modified Polynucleotides. I. Investigation of the Enzymatic Polymerization of 5-alkyl-dUTP-s. Nucleic Acids Res. 1977; 4:2767-2777.
[37] Frydman R B, Valasinas A, Frydman B. Chemical and Enzymatic Polymerization of 2-aminomethyl-3,3′-carboxymethyl-4,4′-(β-carboxyethyl)dipyrrylmethane. Biochemistry 1973; 12:80-85.
[38] Kulshrestha A S, Gao W, Gross R A. Glycerol Copolyesters: Control of Branching and Molecular Weight Using a Lipase Catalyst. Macromolecules 2005; 38:3193-3204.
[39] Mei Y, Kumar A, Gao W, Gross R, Kennedy S B, Washburn N R, Amis E J, Elliott J T. Biocompatibility of Sorbitol-Containing Polyesters. Part I: Synthesis, Surface Analysis and Cell Response in Vitro. Biomaterials 2004; 25:4195-4201.
[40] Gross R A, Kalra B, Kumar A. Polyester and Polycarbonate Synthesis by in Vitro Enzyme Catalysis. Appl. Microbiol. Biotechnol. 2001; 55:655-660.
[41] Srivastava R K, Albertsson A-C. Enzyme-Catalysed Ring-Opening Polymerization of Seven-Membered Ring Lactones Leading to Terminal-Functionalized and Triblock Polyesters. Macromolecules 2006; 39:46-54.
[42] Kumar R, Tyagi R, Parmar V S, Samuelson L A, Watterson A C, Kumar J. Candida antartica Lipase B Catalyzed Copolymerizations of Non-Proteinogenic Amino Acids and Poly(Ethylene Glycol) to Generate Novel Functionalyzed Polyesters. J. Macromol. Sci. 2003; A40:1283-1293.
[43] Kumar R, Shakil N A, Chen M-H, Parmar V S, Samuelson L A, Kumar J, Watterson A C. Chemo-Enzymatic Synthesis and Characterization of Novel Functionalized Amphiphilic Polymers. J. Macromol. Sci. 2002; A39:1137-1149.
[44] Xu P, Uyama H, Whitten J E, Kobayashi S, Kaplan D L. Peroxidase-Catalyzed In Situ Polymerization of Surface Orientated Caffeic Acid. J. Am. Chem. Soc. 2005; 127:11745-11753.
[45] Chi Y S, Jung Y H, Choi I S, Kim Y-G. Surface-Initiated Growth of Poly d(A-T) by Taq DNA Polymerase. Langmuir 2005; 21:4669-4673.
[46] Kim Y-R, Paik H-J, Ober C K, Coates G W, Batt C A. Enzymatic Surface-Initiated Polymerization: A Novel Approach for the In Situ Solid-Phase Synthesis of Biocompatible Polymer Poly(3-hydroxybutyrate). Biomacromolecules 2004; 5:889-894.
[47] Yoon K R, Lee K-B, Chi Y S, Yun W S, Joo S-W, Choi I S. Surface-Initiated, Enzymatic Polymerization of Biodegradable Polyesters. Adv. Mater. 2003; 15:2063-2066.
[48] Yokozawa T, Yokoyama A. Chain-Growth Polycondensation for Well-Defined Condensation Polymers and Polymer Architecture. Chem. Rec. 2005; 5:47-57.
[49] Yokozawa T, Yokoyama A. Chain-Growth Polycondensation: Living Polymerization Nature in Polycondensation and Approach to Condensation Polymer Architecture. Polym. J. 2004; 36:65-83.
[50] Loos K, Muller A H E. New Routes to the Synthesis of Amylose-block-polystyrene Rod-Coil Block Copolymers. Biomacromolecules 2002; 3:368-373.
[51] Oguchi T, Wakisaka A, Tawaki S-I, Tonami H, Uyama H, Kobayashi S. Self-Association of m-Cresol in Aqueous Organic Solvents: Relation to Enzymatic Polymerization Reaction. J. Phys. Chem. B. 2002; 106:1421-1429.
[52] Liu W H, Wang J D, Ma L, Liu X H, Sun X D, Cheng Y H, Li T J. Enzymatic Polymerization of p-phenylphenol in Aqueous Micelles. New York, N.Y.: Acad. Sci. 1995; 750:138-145.
[53] Weissbach H, Pestka S. Molecular Mechanism of Protein Biosynthesis. New York, N.Y.: Academic Press, 1977.
[54] Bermek E. Mechanism of Protein Synthesis. Structure-Function Relations, Control Mechanisms, and Evolutionary Aspects. New York, N.Y.: Springer-Verlag, 1985.
[55] Kornberg A. Biologic Synthesis of Deoxyribonucleic Acid. Science 1960; 131:1503-1508.
[56] Travers A. RNA Polymerase Specificity and the Control of Growth. Nature 1976; 263:641-646.
[57] Light D R, Dennis M S. Purification of a Prenyltransferase That Elongates Cis-polyisoprene Rubber from the Latex of Hevea brasiliensis. J. Biol. Chem. 1989; 264:18589-18597.
[58] Light D R, Lazarus R A, Dennis M S. Rubber Elongation by Farnesyl Pyrophosphate Synthases Involves a Novel Switch in Enzyme Stereospecificity. J. Biol. Chem. 1989; 264:18598-18607.
[59] Dennis M S, Light D R. Rubber Elongation Factor from Hevea brasiliensis. Identification, Characterization, and Role in Rubber Biosynthesis. J. Biol. Chem. 1989; 264:18608-18617.
[60] Eng A H, Ong E L. Hevea Natural Rubber. In: Bhowmick A K, Stephens H L eds. Plastics Engineering; vol 61. Handbook of Elastomers. New York, N.Y.: Marcel Dekker, 2000. p. 29-59.
[61] McIntyre D, Stephens H L, Schloman Jr W W, Bhowmick A K. Guayule Rubber. In: Bhowmick A K, Stephens H L eds. Plastics Engineering; vol 61. Handbook of Elastomers. New York, N.Y.: Marcel Dekker, 2001. p. 1-27.
[62] Puskas J E. In: Producers and World Market of Synthetic Rubbers. In: Koyama E, Steinbüchel A eds. Biopolymers, Vol 2: Polyisoprenoids. Weinheim, Germany: Wiley-VCH, 2001. p. 287-320.
[63] Cornish K. Hypoallergenic Natural Rubber Products from Parthenium argentatum (gray) and other non-Hevea brasiliensis Species. U.S. Pat. Nos. 5,580,942, 1996; 5,717,050, 1998.
[64] Hosier D, Burkett S L, Tarkanian M J. Prehistoric Polymers: Rubber Processing in Ancient Mesoamerica. Science 1999; 284:1988-1991.
[65] Nyburg S C. A Statistical Structure for Crystalline Rubber. Acta. Cryst. 1954; 7:385-392.
[66] Tanaka Y. Structure and Biosynthesis Mechanism of Natural Polyisoprene. Prog. Polym. Sci. 1989; 14:339-371.
[67] Swanson C L, Buchana R A, Otey F H. Molecular Weights of Natural Rubbers from Selected Temperate Zone Plants. J. Appl. Polym. Sci. 1979; 23:743-748.
[68] Castillón J, Cornish K. Regulation of Initiation and Polymer Molecular Weight of Cis-1,4-polyisoprene Synthesized in vitro by Particles Isolated from Parthenium argentatum (Gray). Phytochemistry 1999; 51:43-51.
[69] Cornish K, Castillón J, Scott D J. Rubber Molecular Weight Regulation, in vitro, in Plant Species that Produce High and Low Molecular Weights in vivo. Biomacromolecules 2000; 1:632-641.
[70] Subramaniam A, The chemistry of natural rubber latex. Immun. allergy clinics of N. A. 1995:15, 1.
[71]Cornish K, Biochemistry of natural rubber, a vital raw material, emphasizing biosynthetic rate, molecular weight and compartmentalization, in evolutionarily divergent plant species (1963 to 2000). Nat. Prod. Rep. 2001:18, 182.
[72] Asawatreratanakul K, Zhang Y, Wititsuwannakul D, Wititsuwannakul R, Takahashi S, Rattanapittayaporn A, Koyama T, Molecular Cloning, Expression and Characterization of cDNA Encoding cis-prenyltransferases from Hevea brasiliensis. A Key Factor Participating in Natural Rubber Biosynthesis. Eur. J. Biochem. 2003:270, 4671.
[73] Tanaka Y, Structural Characterization of Natural Polyisoprenes: Solve the Mystery of Natural Rubber Based on Structural Study. Rubber Chem. Technol. 2001:74, 355.
[74] Angulo-Sanchez J L, Cabellero-Mata P, Long Chain Branching in Natural Hevea Rubber—Determination by Gel Permeation Chromatography. Rubber Chem. Technol. 1981:54, 34.
[75] Fuller K N G, Fulton W S, The Influence of Molecular Weight Distribution and Branching on the Relaxation Behavior of Uncrosslinked Natural Rubber. Polymer 1990:31, 609.
[76] Fulton W S, Groves S A, Determination of the Molecular Architecture of Synthetic and Natural Rubber by the Use of Thermal Field-Flow Fractionation and Multi-Angle Laser Light Scattering. J. Nat. Rubber Research 1997:12, 154.
[77] Tangpakdee J, Tanaka Y, Characterization of Sol and Gel in Hevea Natural Rubber. Rubber Chem. Technol. 1997:70, 707.
[78] Tarachiwin L, Sakdapipanich J, Ute K, Kitayama T, Bamba T, Fukusaki E I, Kobayashi A, Tanaka Y. Structural Characterization of alpha-Terminal Group of Natural Rubber. 1. Decomposition of Branch-Points by Lipase and Phosphatase Treatments. Biomacromolecules 2005:6, 1851.
[79] Tarachiwin L, Sakdapipanich J, Ute K, Kitayama T, Tanaka Y. Structural Characterization of alpha-Terminal Group of Natural Rubber. 2. Decomposition of Branch-Points by Phospholipase and Chemical Treatments. Biomacromolecules 2005:6, 1858.
[80] Morton M ed. Rubber Technology. New York, N.Y.: Van Nostrand-Reinhold Co, 1973.
[81] Puskas J E. Diene-Based Elastomers. In: Bhowmick A K, Stephens H L eds. Plastics Engineering; vol 61. Handbook of Elastomers. New York, N.Y.: Marcel Dekker, 2000. p. 817-833.
[82] Gao W, Cui D, Highly cis-1,4 Selective Polymerization of Dienes with Homogenous Ziegler-Natta Catalyst Based on NCN-Pincer Rare Earth Metal Dichloride Precursors, J. AM. Chem. Soc., 2008:130, 4984-4991
[83] Fischbach A, Meermann C, Eickerling G. Discrete Lanthanide Aryl(alk)oxide Trimethylaluminum Adducts as Isoprene Polymerization Catalysts, Macromolecules 2006:39, 6811-6816
[84] Kaita S, Doi Y, Kaneko K. An Efficient Gadolinium Metallocene-Based Catalyst for the Synthesis of Isoprene Rubber, Marcomolecules, 2004:37, 5860-5862
[85] Fujiwara S, Yamanaka A. Efficient Synthesis of trans-polyiosprene compounds using two thermostable enzymes. Biochemical and Biophysical Research Communications, 365, 118-123, 2008
[86] Van G J. Synthesis and Characterization of Polyisoprene with High Cis-content. Adv. Chem. Ser. 1966; 52:136-140.
[87] Morton M. Anionic Polymerization: Principles and Practice. New York, N.Y.: Academic Press, 1983.
[88] Hsieh H L, Quirk R. Anionic Polymerization: Principles and Practical Applications. New York, N.Y.: Marcel Dekker, 1996.
[89] Duck E W, Locke J M. Polydienes by Anionic Catalysts. In: Saltman W M ed. Stereo Rubbers. New York, N.Y.: Wiley Interscience, 1977.
[90] LaFlair, R T. Polybutadiene and Polyisoprene Rubber (section 3.2.1.). In: Ullmann F ed. Ullmann's Encyclopedia of Industrial Chemistry, 6^{th} Edition. Rubber, 3. Synthetic. Weinheim, Germany: Wiley-VCH Verlag, 1998. p. 40-46.
[91] Tanaka Y, Aik-Hwee E, Ohya N, Nishiyama N, Tangpakdee J, Kawahara S, Wititsuwannakul R. Initiation of Rubber Biosynthesis in Hevea brasiliensis: Characterization of Initiating Species by Structural Analysis. Phytochemistry 1996; 41:1501-1505.
[92] Cornish K. The Separate Roles of Plant cis and trans Prenyl Transferases in cis-1,4-polyisoprene Biosynthesis. Eur. J. Biochem. 1993; 218:267-271.
[93] Cornish K, Backhaus R A. Rubber Transferase Activity in Rubber Particles of Guayule. Phytochemistry 1990; 29:3809-3813.
[94] Madhavan S, Greenblatt G A, Foster M A, Benedict C R. Stimulation of Isopentenyl Pyrophosphate Incorporation into Polyisoprene in Extracts from Guayule Plants (Parthenium argentatum Gray) by Low Temperature and 2-(3,4-dichlorophenoxy)triethylamine. Plant Physiol. 1989; 89:506-511.
[95] Archer B L, Audley B G, Cockbain E G, McSweeney G P. The Biosynthesis of Rubber. Incorporation of Mevalonate and Isopentenyl Pyrophosphate into Rubber by Hevea brasiliensis Latex Fractions. Biochem. J. 1963; 89:565-574.
[96] Cornish K, Siler D J. Characterization of cis-prenyl Transferase Activity Localized in a Buoyant Fraction of Rubber Particles from Ficus elastica Latex. Plant Physiol. Biochem. 1996; 34:377-384.
[97] Cornish K, Wood D F, Windle J J. Rubber Particles from Four Different Species, Examined by Transmission Electron Microscopy and Electron-Paramagnetic-Resonance Spin Labeling, are Found to Consist of a Homogeneous Rubber Core Enclosed by a Contiguous, Monolayer Biomembrane. Planta. 1999; 210:85-96.
[98] Cornish K. Similarities and Differences in Rubber Biochemistry among Plant Species. Phytochemistry 2001; 57:1123-1134.
[99] Cornish K. Private Communication.
[100] Archer B L, Audley B G. New Aspects of Rubber Biosynthesis. Bot J Linn Soc 1987; 94:181-196.
[101] Dubey V S, Bhalla R, Luthra R. An Overview of the Non-Mevalonate Pathway for Terpenoid Biosynthesis in Plants. J Biosci 2003; 28:637-646.
[102] Scott D J, Da Costa B M T, Espy S C, Keasling J D, Cornish K. Activation and Inhibition of Rubber Transferases by Metal Cofactors and Pyrophosphate Substrates. Phytochemistry 2003; 64:123-134.
[103] Ownby D R, Ownby H E, McCullough J, Shafer A W. The Prevalence of Anti-Latex IgE Antibodies in 1000 Volunteer Blood Donors. J. Allergy Clin. Immunol. 1996; 97:1188-1192.
[104] Pailhories G. Reducing Proteins in Latex Gloves. The industrial approach. Clin. Rev. Allergy 1993; 11:391-402.
[105] Tomazic V J, Withrow T J, Fisher B R, Dillard S F. Latex-Associated Allergies and Anaphylactic Reactions. Clin. Immunol. Immunopathol. 1992; 64:89-97.
[106] Morales C, Basomba A, Carreira J, Sastre A. Anaphylaxis Produced by Rubber Glove Contact. Case Reports and Immunological Identification of the Antigens Involved. Clin. Exp. Allergy 1989; 19:425-430.
[107] Slater J E. Rubber Anaphylaxis. N. Engl. J. Med. 1989; 320:1126-1130.
[108] Mooibroek H, Cornish K. Alternative sources of natural rubber. Appl. Microbiol. Biotechnol. 2000; 53:355-65.
[109] Archer B L, Audley B G. Biosynthesis of Rubber. Advances in Enzymology and Related Subjects of Biochemistry 1967; 29:221-257.
[110] Lynen F., Henning U. Biological Path to Natural Rubber. Angew. Chem. 1960:72, 820.
[111] Poulter C D, Satterwhite D M, Rilling H C, Prenyltransferase. The Mechanism of the Reaction. JACS 1976:98, 3376.
[112] Tanaka Y, Kawahara S, Eng A H, Shiba K, Ohya N. Initiation of biosynthesis in Cis-polyisoprenes. Phytochemistry 1995: 39, 779
[113] Xie W, McMahan C M, DeGraw A J, Distefano M D, Cornish K, Whalen M C, Shintani D K, Initiation of rubber biosynthesis: In vitro comparisons of benzophenone-modified diphosphate analogues in three rubber-producing species. Phytochemistry 2008:69, 2539.
[114] Mau C J D, Scott D J, Cornish K, Multiwell filtration system results in rapid, high-throughput rubber transferase microassay. Phytochem. Anal. 2000:11, 356.
[115] Benedict C R, Madhavan S, Greenblatt G A, Venkatachalam K V, Foster M A, The enzymic synthesis of rubber polymer in Parthenium argentatum Gray. Plant Physiol. 1990:92, 816.
[116] Tangpakdee J, Tanaka Y, Ogura K, Koyama T, Wititsuwannakul R, Wititsuwannakul D, Rubber formation by fresh bottom fraction of Hevea latex. Phytochemistry 1997:45, 269.
[117] Tangpakdee J, Tanaka Y, Ogura K, Koyama T, Wititsuwannakul R, Wititsuwannakul D, Asawatreratanakul K, In vitro synthesis of Hevea rubber by bottom fraction and C-serum. Part 1. Isopentenyl diphosphate isomerase and prenyl transferase activities in bottom fraction and C-serum from Hevea latex. Phytochemistry 1997:45, 261.
[118] Tangpakdee J, Tanaka Y, Ogura K, Koyama T, Wititsuwannakul R, Chareonthiphakorn N, Structure of in vitro synthesized rubber from fresh bottom fraction of Hevea latex. Phytochemistry 1997:45, 275.
[119] Wititsuwannakul D, Rattanapittayaporn A, Wititsuwannakul R, Rubber biosynthesis by a Hevea latex bottom-fraction membrane. Journal of Applied Polymer Science 2003:87, 90.
[120] Wititsuwannakul D, Wititsuwannakul R. “Polyisoprenoids,”, Wiley Weinheim, 2001.
[121] Tanaka Y, Aik-Hwee E, Ohya N, Nishiyama N, Tangpakdee J, Kawahara S, Wititsuwannakul R, Initiation of rubber biosynthesis in Hevea brasiliensis: characterization of initiating species by structural analysis. Phytochemistry 1996:41, 1501.
[122] McMullen A I. The Polynucleotide Double Helix as a Versatile Assembly Template during Polymer Biogenesis. J Theoret Biol 1963; 5:127-141.
[123] Kaneko Y, Kadokawa J-I. Vine-Twining Polymerization: a New Preparation Method for Well-Defined Supramolecules Composed of Amylose and Synthetic Polymers. Chem. Rec 2005; 5:36-46.
[124] Mejias L, Schollmeyer D, Sepulveda-Boza S, Ritter H. Cyclodextrins in Polymer Synthesis: Enzymatic Polymerization of a 2,6-dimethyl-β-cyclodextrin/2,4-dihydroxyphenyl-4′-hydroxybenzylketone Host-Guest Complex Catalyzed by Horseradish Peroxidase (HRP). Macromol Biosci 2003; 3:395-399.
[125] Minetti C A S A, Remeta D P, Miller H, Gelfand C A, Plum G E, Grollman A P, Breslauer K J. The Thermodynamics of Template-Directed DNA Synthesis: Base Insertion and Extension Enthalpies. Proc Natl Acad Sci USA 2003; 100:14719-14724.
[126] Kadokawa J-I, Kaneko Y, Nagase S-I, Takahashi T, Tagaya H. Vine-Twining Polymerization: Amylose Twines Around Polyethers to Form Amylose-Polyether Inclusion Complexes. Chemistry 2002; 8:3321-3326.
[127] Sahoo S K, Nagarajan R, Chakraborty S, Samuelson L A, Kumar J, Cholli A L. Variation in the Structure of Conducting Polyaniline with and without the Presence of Template during Enzymatic Polymerization: a Solid-State NMR Study. J Macromol Sci, Pure Appl Chem 2002; A39:1223-1240.
[128] Kadokawa J-I, Kaneko Y, Nakaya A, Tagaya H. Formation of an Amylose-Polyester Inclusion Complex by Means of Phosphorylase-Catalyzed Enzymatic Polymerization of α-D-Glucose 1-Phosphate Monomer in the Presence of Poly(ε-caprolactone). Macromolecules 2001; 34:6536-6538.
[129] Puskas J E, Chan S W P, McAuley K B, Shaikh S, Kaszas G. Kinetics and Mechanisms in Carbocationic Polymerisation: the Quest for True Rate Constants. J Polym Sci 2005; 43:5394-5413.
[130] Puskas J E, Kaszas G. Carbocationic Polymerization. In: Kroschwitz J I ed. Encyclopedia of Polymer Science and Technology, V5. New York, N.Y.: Wiley-Interscience, 2003. p. 382-418.
[131] Matyjaszewski K, Sawamoto M. Cationic Polymerizations: Mechanisms, Synthesis, and Application. In: Matyjaszewski K ed. Plastics Engineering. New York, N.Y.: Marcel Dekker Inc, 1996. vol 35 p. 343-375.
[132] Poulter C D, Rilling H C. The Prenyl Transfer Reaction. Enzymatic and Mechanistic Studies of the 1′-4 Coupling Reaction in the Terpene Biosynthetic Pathway. Acc Chem Res1978; 11:307-313.
[133] Kellogg B A, Poulter C D. Chain Elongation in the Isoprenoid Biosynthetic Pathway. Curr Opin Chem Biol 1997; 1:570-578.
[134] Harris C M, Poulter C D. Recent Studies of the Mechanism of Protein Prenylation. Nat. Prod Rep, Royal Soc Chem 2000; 17:137-140.
[135] Liang P-H, Ko T-P, Wang A H-J. Structure, Mechanism and Function of Prenyltransferases. Eur J Biochem 2002; 269:3339-3354.
[136] Satoh K, Kamigaito M, Sawamoto M. Sulfonic Acids as Water-Soluble Initiators for Cationic Polymerization in Aqueous Media With Yb(OTf)₃. J Polym Sci, Part A: Polym Chem 2000; 38:2728-2733.
[137] Satoh K, Kamigaito M, Sawamoto M. Metal Triflates and Tetrafluoroborates as Water-Tolerant Lewis Acids for Cationic Polymerization in Aqueous Media. Macromolecules 2000; 33:5836-5840.
[138] Hoffmann M, Hampel N, Kanzian T, Mayr H. Electrophilic Alkylations in Neutral Aqueous or Alcoholic Solutions. Angew Chem 2004; 43:5402-5405.
[139] Shaikh S, Puskas J E, Kaszas G. A New High-Throughput Approach to Measure Copolymerization Reactivity Ratios using Real-Time FTIR Monitoring. J Polym Sci Chem 2004; 42:4084-4100.
[140] Kaszas G, Puskas J E, Kennedy J P. New Thermoplastic Elastomers of Rubbery Polyisobutylene and Glassy Cyclopolyisoprene Segments. J Appl Polym Sci 1990; 39:119-144.
[141] Puskas J E, Gautriaud E, Deffieux A, Kennedy J P, Natural rubber biosynthesis-A living carbocationic polymerization? Progress in Polymer Science 31[6], 533-548, 2006

### SUMMARY OF THE INVENTION

An in vitro process produces cis-1,4-polyisoprene or homolog polymers by utilizing washed rubber particles WPR from natural rubber latex, or the latex itself, that inherently contain an enzyme catalyst desirably having a cofactor component, isoprene or other diene monomers, and various allylic initiators that effect a carbocationic-like (electrophilic, and preferably living) polymerization of the isoprene or other synthetic monomers. Optionally the reaction can be carried out in the presence of pyrophosphoric acid. The initiators are generally allylic pyrophosphates or functionalized allylic pyrophosphates. The enzyme is typically a rubber enzyme such as prenyl transferase. The polymerization temperature can vary over a wide range as well as the number average molecular weight of the produced cis-1,4-polyisoprene. The polymers produced are essentially free or completely free of traditional synthetic catalysts used in rubber production processes such as coordination catalysts containing trialkyl aluminum and titanium tetrachloride, or a catalyst of an aluminum hydride derivative and titanium tetrachloride, or lithium alkyl and hence does not possess any adverse effects thereof. The properties of the polymer produced from isoprene monomer should be close or equivalent to that of natural rubber. By the term “essentially free of” it is meant that for every 100 parts by weight of polymer the amount of non-enzyme catalysts therein is less than about 10 ppm, desirably less than about 5 ppm, and preferably less than about 1 ppm by weight.

A process for the synthetic production of cis-1,4-polyisoprene or cis-1,4-polydiene, comprising the steps of: mixing, in any order, isoprene monomers or other diene monomers having from 4 to 8 carbon atoms, a natural rubber latex or washed rubber particles (WRP), and an allylic initiator, said latex or WRP containing an enzyme catalyst and a divalent cofactor therein; and polymerizing said mixture and producing cis-1,4-polyisoprene or cis-1,4-polydiene.

A process for the in vitro production of natural rubber comprising the steps of: adding an allylic initiator and isoprene monomers or one or more other diene monomers having from 4 to 8 carbon atoms to a natural rubber latex or washed rubber particles containing an enzyme catalyst and optionally a divalent cofactor; and carbocationically or electrophilically polymerizing said isoprene or said one or more other diene monomers, optionally in the presence of pyrophosophoris acid or salts thereof, to produce cis-1,4-polyisoprene or cis-1,4-polydiene.

A natural rubber composition comprising: polymers of cis-1,4-polydienes other than cis-1,4-polyiosprene that are essentially free of non-enzyme catalysts therein.

A process for the in vitro production of isoprenoids, comprising the steps of: forming a solution comprising an allylic pyrophosphate initiator, isoprene or other diene monomers containing from 4 to 8 carbon atoms, an enzyme catalyst and optionally a cofactor; and carbocationically polymerizing said isoprene and said other diene monomers to produce a polyisoprenoid composition.

### BRIEF DESCRIPTION OF THE DRAWINGS

FIG. 1 is a visualization of Yokozawa's concept of chain-growth polycondensation [48,49];

FIG. 2 is an attempted “bio-inspired” synthesis of cPIP [48];

FIG. 3 is the microstructure of natural Hevea rubber [66];

FIG. 4 relates to a visualization of natural rubber particles and their structure;

FIG. 5 relates to the deproteinization by treatment with 1˜2% ethanol;

FIG. 6 is possible enchainments in polyisoprene;

FIG. 7 is the structure of IPP at pH=7, and initiator formation in NR biosynthesis;

FIG. 8 shows the structures of allylic oligoisoprene pyrophosphates (APPs): geranyl-, farnesyl- and geranyl-geranyl-pyrophosphates (GPP, FPP and GGPP respectively);

FIG. 9 is a natural rubber biosynthesis in biochemical symbolism [69];

FIG. 10 is a natural rubber biosynthesis. a. Initiation, b. Propagation, in polymer chemical symbolism [66];

FIG. 11 relates to SEC trace of bottom fraction (dash line) and in vitro synthesized natural rubber (solid line);

FIG. 12 shows the mechanism of polyisoprene formation proposed by Archer [109];

FIG. 13 shows proposed initiation mechanism in NR biosynthesis (En=enzyme);

FIG. 14 shows possible role of divalent cations in NR biosynthesis;

FIG. 15 shows “Ionization-Condensation-Elimination” mechanism proposed by Poulter et al;

FIG. 16 shows isomers obtained by proton loss from a tertiary carbocation;

FIG. 17 shows a scheme of the active sites in avian trans-prenyltransferase;

FIG. 18 shows the termination: hydrolysis of the chain end;

FIG. 19 shows the proposed “NLCP” mechanism;

FIG. 20 relates to comparing the LS and RI traces of the WRP-1/5(IPP) and WRP-1/24(IPP) with WRP-1;

FIG. 21 relates to comparing the LS and RI traces of the 5 and 24 hr samples using IP as monomer with WRP-1;

FIG. 22 relates to comparing the LS and RI traces of the 5 and 24 hr samples using IP as monomer in the presence of 10% (v/v) ethanol with WRP-1;

FIG. 23 shows the Raman spectra of pure IAC40 WRP;

FIG. 24 shows a 3D time progression Raman spectrum of IAC40 WRP upon addition of isoprene monomer;

FIG. 25 shows the growth of polyisoprene mode over six hours;

FIG. 26 shows SEC of IAC40 latex, and IAC40 latex (IP/30).

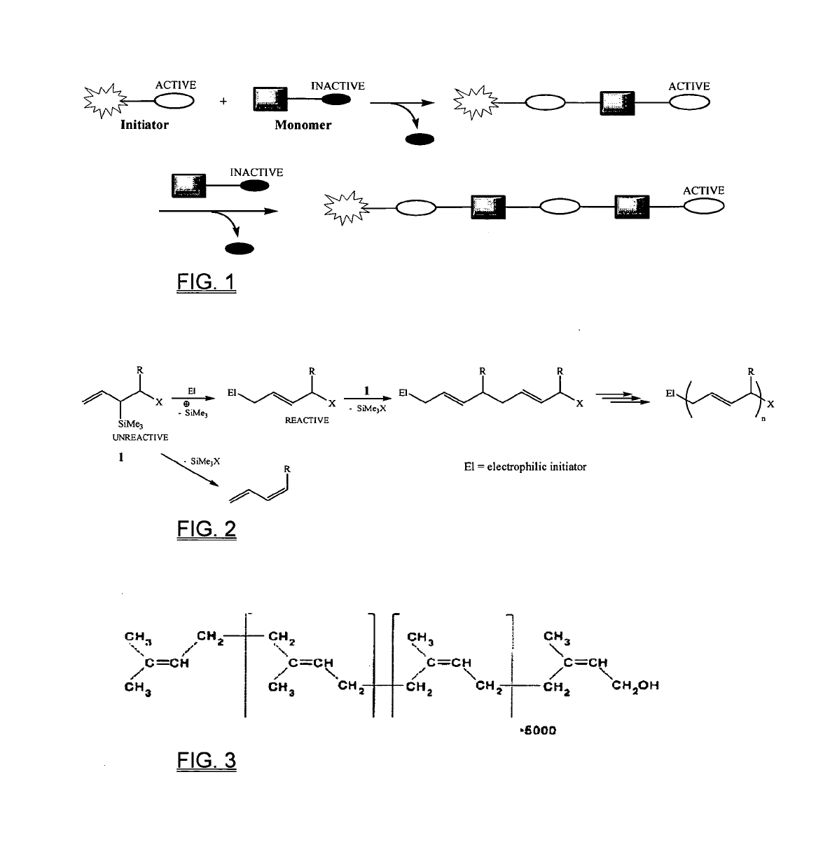

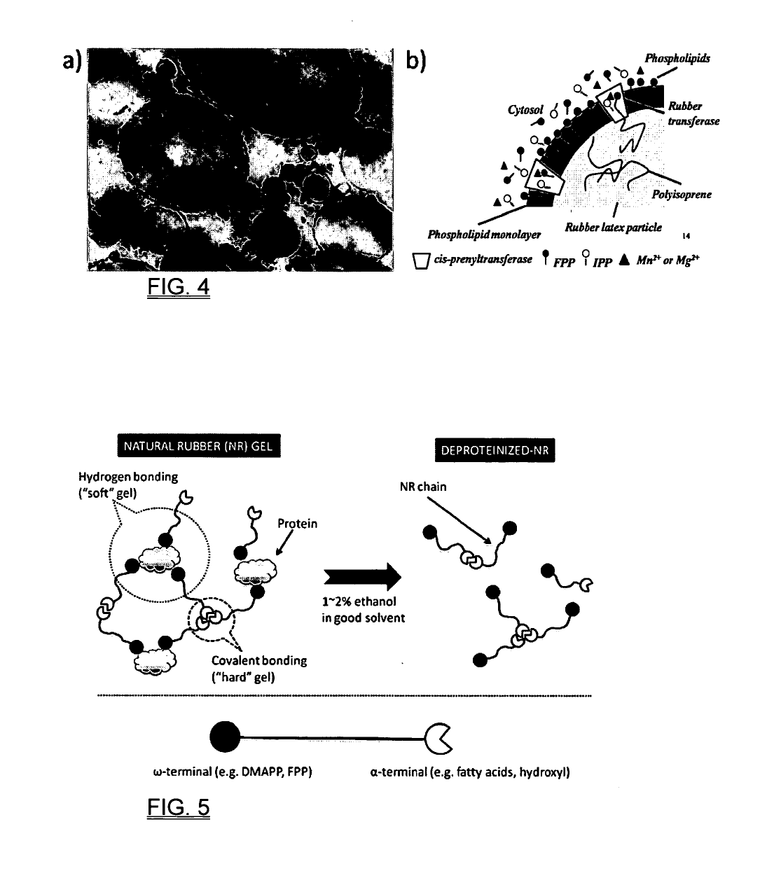

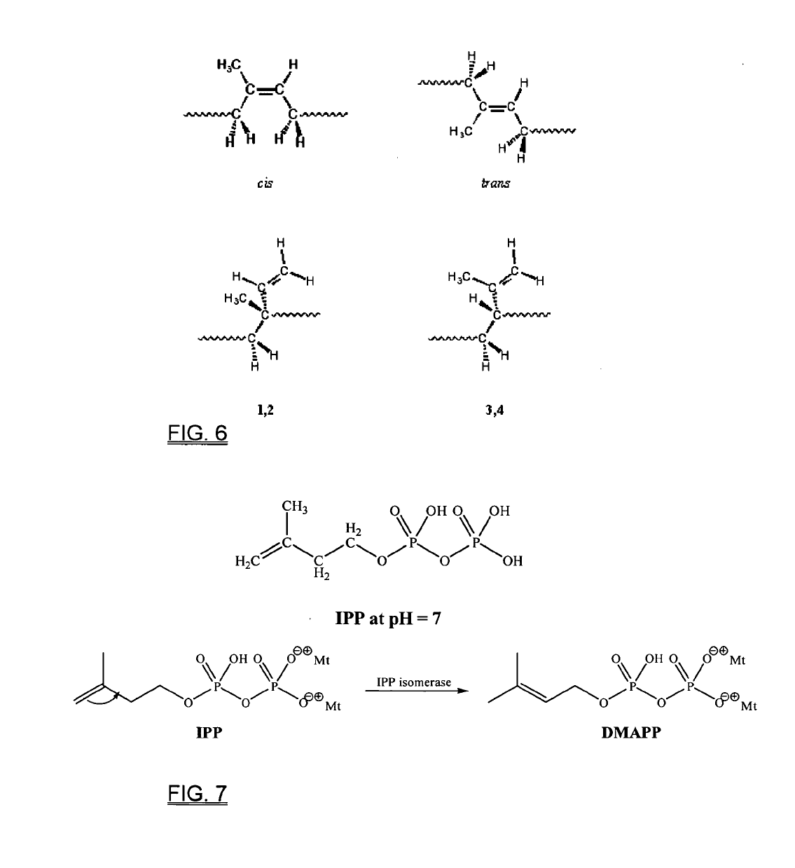

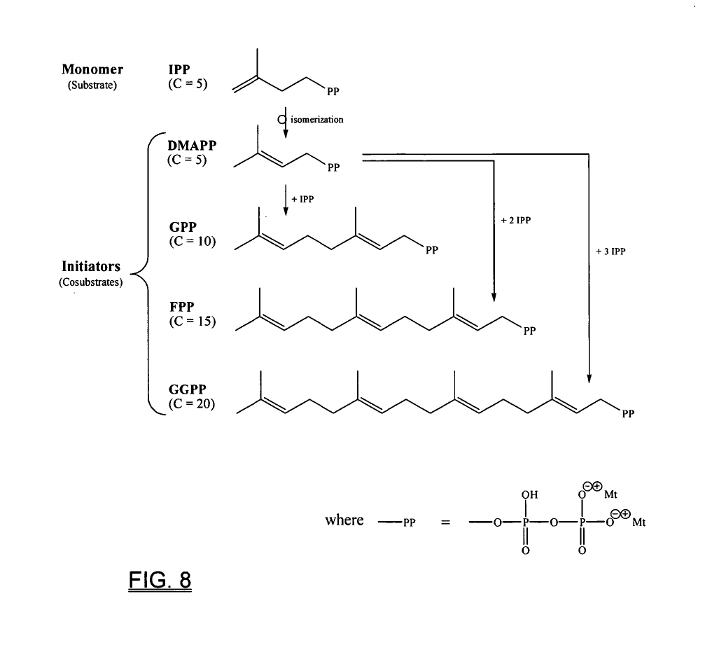

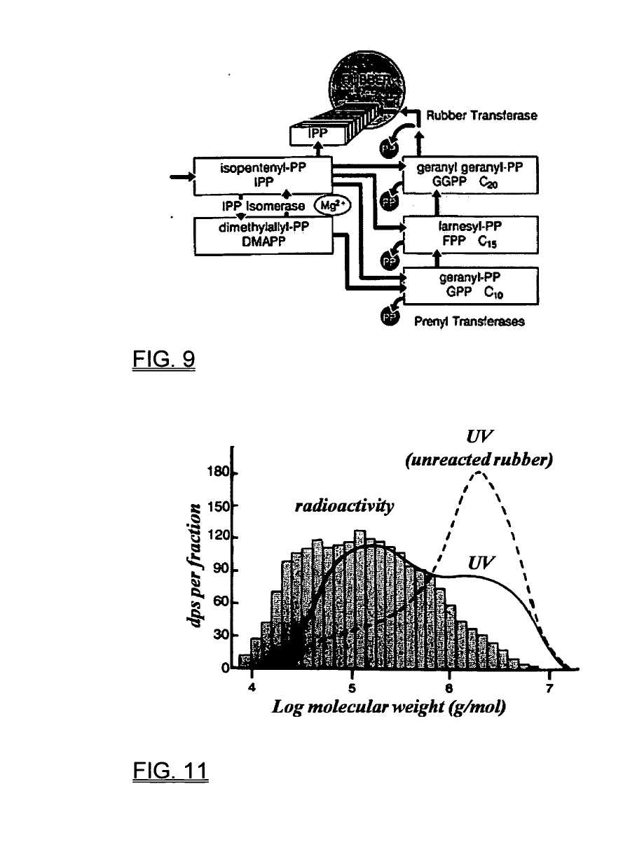

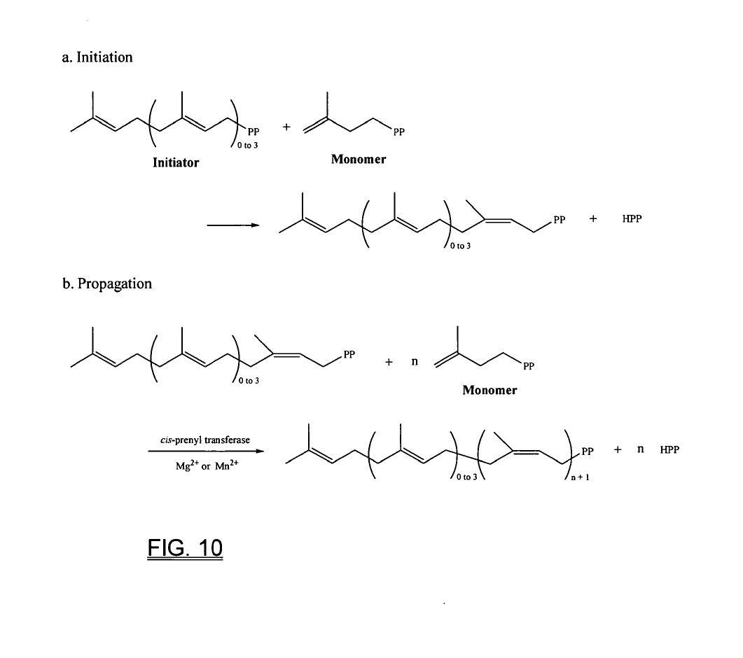

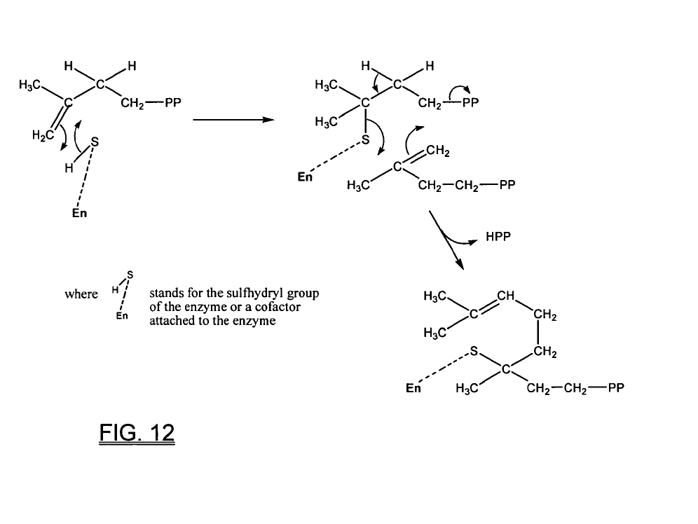

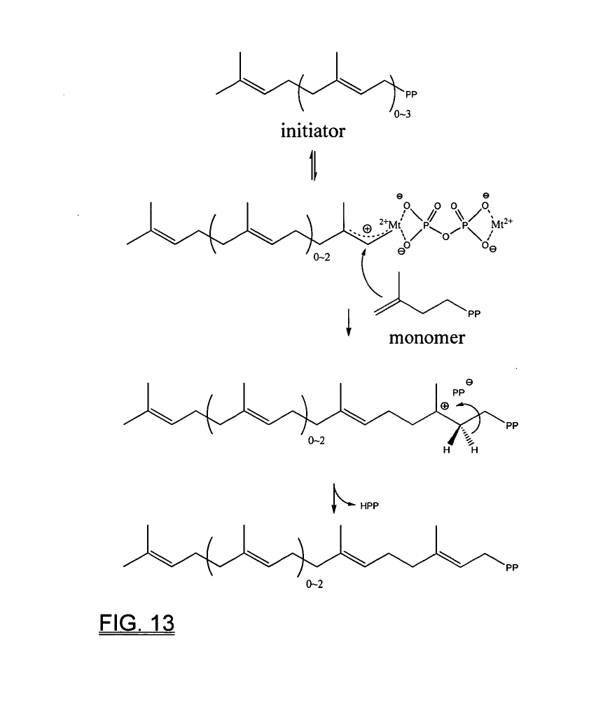

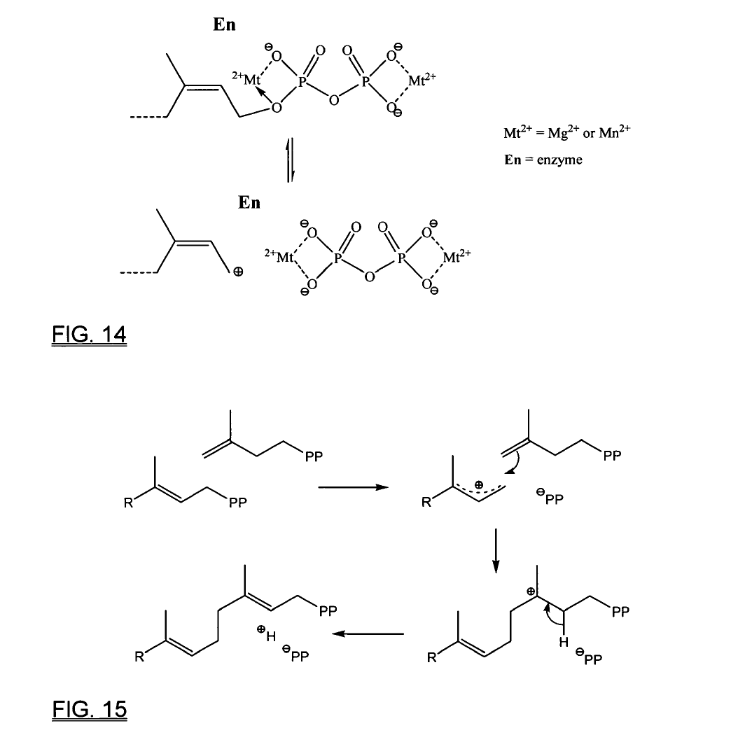

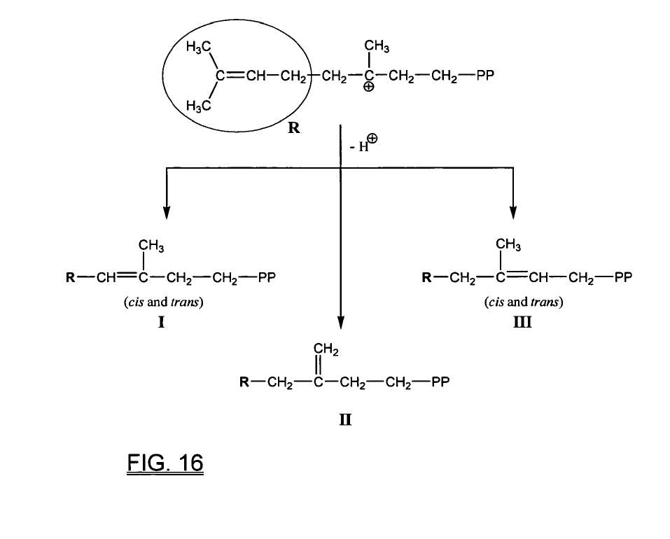

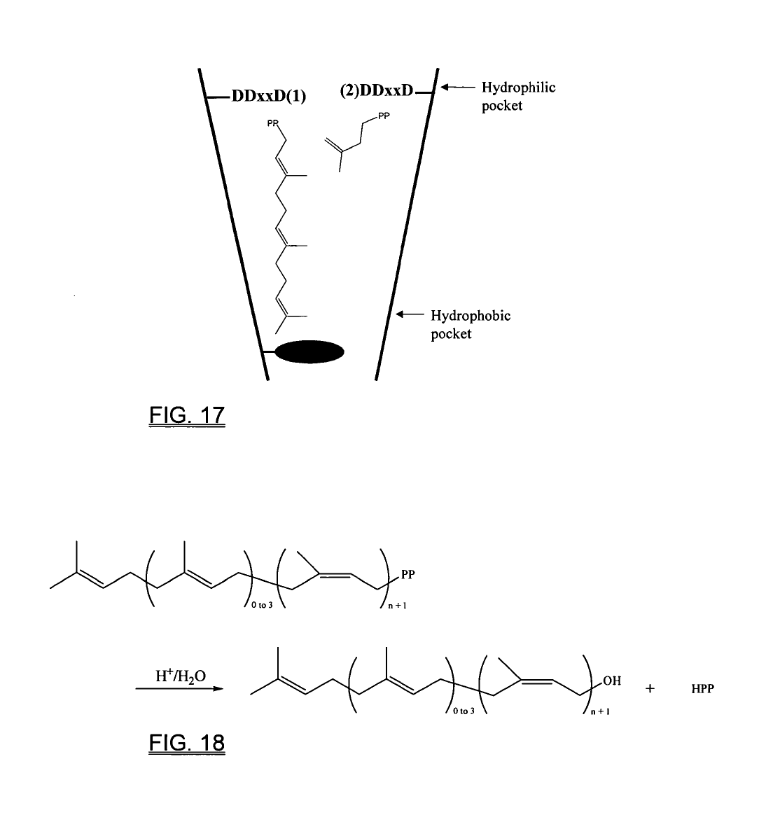

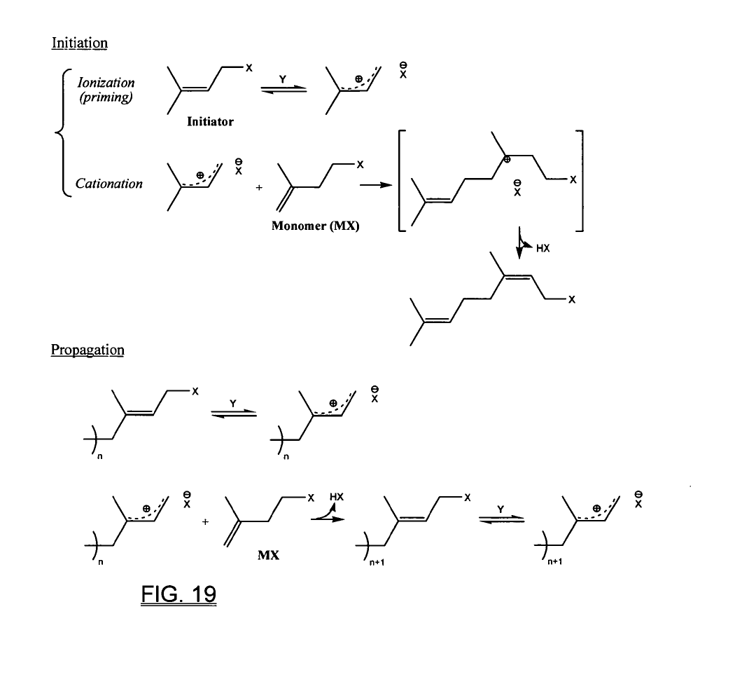

### DETAILED DESCRIPTION OF THE INVENTION

Heretofore, natural rubber has been obtained from various sources such as primarily the Brazilian rubber tree (Hevea brasiliensis) as well as other plants and shrubs as set forth in Table 1.

TABLE 1

| | | Rubber- | Rubber | |
|---|---|---|---|---|
| Species | Family | producing tissues | Harvest | Characteristics |
| Hevea brasiliensis | Euphorbiaceae | Many, collected | 30-50% of | Perennial, |
| (Brazilian Rubber Tree) | | from outer bark | exuded latex | narrow genetic diversity |
| Parthenium argentatum | Asteraceae | Bark | 5-10% | Perennial, |
| (guayule desert shrub) | | parenchyma | plant dw | Varieties bred for |
| | | | | rubber production |
| Helianthus annuus | Asteraceae | Primarily in | Up to 1% | Annual, |
| (Sunflower) | | leaves | leaf dw | Many cultivars, no |
| | | | | rubber selection |
| Taraxacum kok-saghyz | Asteraceae | Primarily in roots | Up to 15% | Annual (can grow 2 years) |
| (Russian dandelion) | | phloem parenchyma | of root dw | Essentially wild type |
| Ficus elastica | Moraceae | Stems & leaves | ~25% of | Perennial, |
| (India rubber fig) | | | exuded latex | Bred for ornamental use |
| Lactuca serriola, | Asteraceae | Stems & leaves | <1% plant dw | Annual, |
| Lactuca sativa | | | | Many cultivars, no |
| (Lettuce) | | | | rubber selection |
| Hancornia speciosa | Apocynaceae | Collected from | 25-30% of | Perennial, grown for |
| (Mangabiera) | | outer bark | exuded latex | fruit |
| Cryptostegia grandiflora | Apocynaceae | Roots, stems, | — | Perennial woody vine, |
| (Madagascar Rubber Vine) | | leaves | | considered invasive |

Depending on the seasonal effects and the state of the soil, the average composition of Hevea NR latex can vary and thus by rough approximation contain 25-35 wt % cis-1,4-polyisoprene; 1˜1.8 wt % protein; 1˜2 wt % carbohydrates; 0.4˜1.1 wt % lipids 0.5˜0.8 wt % amino acids, and 50˜70 wt % water. [70] The latex particles (FIG. 4a) are stabilized by a membrane of phospholipid monolayer (FIG. 4b). [71] The rubber enzyme (cis-prenyltransferase) is a membrane-bound amphiphilic enzyme, yet to be isolated and fully characterized. [72,73] Therefore, in vitro NR biosynthesis relies on the use of active membrane-bound rubber transferase in the so-called “Washed Rubber Particles” (WRP) obtained from the latex by removing the non-rubber constituents, or in the “bottom fraction”. The WRP still contains approximately 6% non-polyisoprene constituents and other non-soluble constituents such as membrane-bound proteins, phospholipids, and the like.

While the examples and some portions of the description of the invention relate to the utilization of latex and WPR from Brazilian rubber trees, it is to be understood that any source of natural rubber including blends of two or more sources of natural rubber can be utilized by the present invention.

According to the present invention, the source of natural rubber latex is washed in any conventional manner known to the art and to the literature. For example, one method relates to washing the latex in a slightly alkaline solution such as T_{RIS}-HCl (Tris(hydroxymethyl)aminomethane Hydrochloride) having a pH of about 7.2 to about 8.0, DTT (dithiothreitol), and AEBSF [4-(2-amnioethyl)benzenesulfonyl fluoride hydrochloride]. Until needed, the purified washed rubber particles can be stored in a polyhydric alcohol such as glycerol in liquid nitrogen. An amount of water is utilized so that the washed latex generally has a solids content of about 5 to about 80 wt. %—specifically about 10 to about 60 wt. %.

More specifically, another method of washing the natural rubber latex can be accomplished as follows: The latex of any of the above noted source of natural rubber, such as Hevea (RRIM600) collected from plantation trees, was stabilized with a buffer solution (0.1M NaHCO₃, 50% glycerol, 0.3% (w/v) NaN₃, 5 mM cysteine), then shipped to a desired location in dry ice and stored at −80° C. until use. The RRIM designation represents that these H. brasiliensis trees were cloned and developed at the Rubber Research Institute of Malaysia and 600 signifies that it is a representative clone of normal H. brasiliensis trees. Tris-HCl (Tris(hydroxymethyl) aminomethane Hydrochloride), dithiothreitol (DTT), [4-(2-aminoethyl)benzenesulfonyl fluoride hydrochloride] (AEBSF) and ethylenediamine-tetraacetic acid (EDTA) can be obtained from Sigma-Aldrich and used as received. Tetrahydrofuran (HPLC grade) can be obtained from Fisher Chemical and distilled over sodium/benzophenone. Polyisoprene standards (>95% cis-1,4 PIP) with Mₙ=2,450 and 9,870 g/mol and M_{w}/Mₙ=1.02 and 1.03 can be purchased from Scientific Polymer Products, Inc. Smoked natural rubber can be provided by the Goodyear Tire & Rubber Co. Subsequently, the active rubber particles from Hevea (RRIM600) latex can be purified by washing a few times, e.g. from 2 to about 10, in 100 mM Tris-HCl (pH 7.5), 5 mM DTT and 0.1 mM AEBSF, centrifugation and suspension in wash buffer. After four washes, 10% glycerol can be added and then the WRPs can be transferred into liquid nitrogen. The wash natural rubber latex can then be utilized as needed.

The washed natural rubber inherently contains a biocatalyst, that is, an enzyme that has been found to catalyze natural rubber biosynthesis in the presence of isoprene and diene monomers. More specifically, the biocatalyst acts together with divalent metal cation cofactors (e.g. groups 2 through 12 of the Periodic table) such as Mg²⁺ (preferred) or Mn²⁺ in order to allow enzymatic activity towards isoprene and diene monomers. The inherent biocatalyst is prenyl transferase. This enzyme catalyst system as well as other related catalyst will catalyze the reaction of the isoprene monomers with a carbocationic initiator (a cationizing agent) discussed herein below. The amount of the enzyme catalyst within the washed natural rubber generally containing a cofactor, mono but preferably multivalent, was found to be sufficient to polymerize large quantities of isoprene monomer. It has also been demonstrated earlier that by adding the cofactor to an enzyme dramatically increases the rate of in vitro polymerization. Therefore it is anticipated that the addition of cofactors will also accelerate isoprene incorporation as defined in the present invention. The amount of the added cofactor, preferably Mg²⁺ is from about 0.1 to about 100 millimoles and preferably from about 0.5 to about 5 millimoles per 100 parts by weight of isoprene monomers.

An important aspect of the present invention is the utilization of allylic initiators that are capable of generating an allylic carbocation to effect carbocationic polymerization of the isoprene monomers. Many allylic compounds can be utilized with a large class thereof being various allylic pyrophosphates or functionalized allylic pyrophosphates or compounds containing an allylic pyrophosphate end group. Examples of such initiators include allylic pyrophosphate, 1,1-dimethylallyl pyrophosphate, farnesyl pyrophosphate, geranyl-geranyl pyrophosphate, geranyl pyrophosphate, allylic neryl pyrophosphate; generally oligomers with an allylic pyrophosphate end group such as oligoisoprenes, and the like. Other allylic initiators include dimethyl allyl halides such as geranyl-, geranyl geranyl-, farnesyl-, neryl etc. bromides, chlorides, iodides, fluorides, as well as various hydroxides such as dimethyl allyl hydroxide, geranyl geranyl-, farnesyl-, neryl and the like. The initiators can carry other functional groups such as an oligoisobutylene head group or a polymerizable group ((meth)acrylate, acrylamide, lactone, lactame, etc), or a protected polymerizable group (protected acid, protected amine, etc). Preferred initiators include farnesyl pyrophosphate, geranyl pyroposphate, and neryl pyrophosphate. The various allylic pyrophosphates and halides and hydroxyls are known to the literature and to the art and sources thereof include any Sigma-Alrich Handbook of Chemicals.

While the washed natural rubber latex can inherently contain suitable allylic initiators therein, it is generally desirable to add allylic initiators or functionalized allylic initiators that can be added separately or with the isoprene monomer to the washed natural rubber latex. The amount of the allylic initiator is generally from about 0.01 to about 10 millimoles and desirably from about 0.1 to about 1 millimoles per 100 parts by weight of the isoprene or other diene monomers.

The in vitro biosynthesis of natural rubber from a washed natural rubber is achieved utilizing preferably isoprene or other diene monomers containing from 4 to 8 carbon atoms. Suitable diene monomers include 1,3-butadiene, 2,3-dimethyl-butadiene and the like. The monomers of the present invention exclude allylic or alkene monomers such as isopentene.

It is a great advantage of the current invention that the isoprene monomers and the various diene monomers do not need to have a high purity, in contrast to synthetic rubber manufacturing processes that require high purity monomers such that the amount of impurities therein, for example various hydrocarbons, is generally less than 1 parts by weight, desirably less than 0.1 parts by weight, and preferably less than 0.01 parts by weight per every 100 parts by weight of isoprene monomers and/or diene monomers. The specificity of the enzymes towards isoprene or dienes allows the use of non-purified isoprene from oil or biomass.

The amount of monomers utilized is such that a washed natural rubber feedstock containing about 5% solids by weight will contain after polymerization of the monomers from about 10% to about 30% by weight of solids therein, and preferably all the added monomers will form rubber.

Various solvents have been found to improve the solubility of the isoprene and/or diene monomers but not to diminish the activity of the inherent natural rubber enzyme catalyst. Such solvents generally include alcohols having from about 1 to about 5 carbon atoms with ethanol being preferred. Organic solvents (alkyl chloride, aromatics, amides, ketones, esters) can also be used. The amount of the solvents generally range from about 4 to about 20 parts by weight and desirably from about 7 to about 15 parts by weight based upon every 100 parts by weight of the isoprene and/or diene monomers.

The carbocationic polymerization of the monomers of the present invention can occur over a wide temperature range such as from about 0° C. to about 60° C., desirably from about 15° C. to about 40° C. with from about 20° C. to about 30° C. being preferred. As noted above, since in this invention the polymerization of the isoprene monomers or other diene monomers is similar to a living polymerization very little or no chain transfer or chain termination occurs. That is, generally less than 10%, desirably less than 5%, and preferably less than 1% of all the polymerizing chains are ended by either chain transfer and/or chain termination. While not desired, it is within the scope and ambit of the present invention that some chain transfer and/or chain termination can occur as by utilizing chain transfer or chain termination agents known to the art and to the literature. Examples of such chain termination agents include cationic traditional terminating agents such as water. Ethanol obviously does not terminate NR biosynthesis. Chain termination can also occur via removing the cofactor(s) by chelation with EDTA.

Polymerization times can vary widely such as from about 3 to about 48 hours. The polymerization desirably is carried out in a closed system and thus pressure of the reaction can range from about 1 to about 2 and desirably from about 1 to about 1.1 atmosphere. It may be also of interest to add an organic cosolvent to increase the concentration of IP. It may be preferable to use an atmosphere containing carbon dioxide at about 5%.

The molecular weight of the cis-1,4-polyisoprene polymers or other cis-1,4-polydiene polymers of the present invention can vary greatly depending upon the amount of allylic initiator utilized. Thus, the number of cis-1,4-isoprene or cis-1,4-diene repeat units can range from about 5 or from about 10 to a very high molecular weight of up to about 20,000, or about 25,000 or about 30,000 repeat units. In the case of natural rubber the number of units is desirably from about 5000 to about 15,000, and preferably from about 8000 to about 10,000. The molecular weight distribution, i.e. M_{w}/Mₙ can vary as from about 2 to about 20 and preferably from 2 to about 5. The broad molecular weight distributions can be obtained by different methods such as by adding the initiators at different stages, and the like. A unique aspect of the present invention is that stereoregular polymers such as cis-1,4-polyisoprene or cis-1,4-polydiene are made that ordinary synthetic catalysts are not capable of producing.

The invention will be better understood by reference to the following examples that serve to illustrate, but not to limit the invention including the in vitro production of natural rubber.

The preparation of the in vitro natural rubber can generally be described as follows: A suitable synthetic allylic initiator that produces an allylic carbocation is added to a natural rubber latex or washed rubber particles that inherently contain a naturally occurring cis-prenyl transferase enzyme with cofactors therein, along with synthetic isopentenyl pyrophosphate IPP or isoprene IP monomers. Desirably the initiator is added first followed by the monomer although the addition can be carried out in any order, e.g. initially adding the monomers followed by the initiator. The initiator at ambient temperature will initiate the polymerization of the monomers in the presence of the enzyme and cofactors until essentially all of the monomer is polymerized. We propose that the polymerization essentially is a living carbocationic (electrophilic) polymerization, but this is not a necessary criterion.

Examples for in vitro rubber biosynthesis. Enzymatically active rubber particles from Hevea (RRIM600) latex were purified by washing four times in 100 mM Tris-HCl (pH 7.5), 5 mM DTT and 0.1 mM AEBSF, centrifugation and suspension in wash buffer. After four washes, 10% glycerol was added and then the WRPs were transferred into liquid nitrogen. These will be designated as Washed Rubber Particles (WRP-1 and WRP-3), due to two different batches of RRIM600 latex. WRP-1 was used in conventional in vitro NR biosynthesis and WRP-3 was used in “large” scale experiments. Most of the in vitro experiments were carried out in wells of 96-well filter plates (Millipore Durapore membrane 0.65 μm). Typically, the reaction volume was 20 μL comprising of 2 μL of buffer (100 mM Tris-HCl, pH 7.5, 1.25 mM MgSO4, 5 mM DTT), 0.4 μL (4×10⁻⁸ mol) of 100 mM IPP or IP water solution/dispersion, 0.6 μL (6×10−8 mol) of 1 mM FPP in water and 2 mg of WRP in 17 μL of water. 5 hrs and 24 hrs reaction times were used at 25° C. in an incubator. The reactions were stopped by adding 40 μL (3.2×10−6 mol) of 80 mM EDTA. The filter plate was vacuumed and then washed two times with 150 μL water then once with 95% ethanol, then oven-dried at 37° C. for 30 minutes.

One larger scale reaction was performed in 3.8 mL buffer (100 mM Tris-HCl, 1.25 mM MgSO4, 5 mM DTT, 0.1 mM AEBSF, pH 7.5) containing 0.1 mM (3.8×10−7 mol) IPP, 15 μM (5.7×10−8 mol) FPP and 76 mg WRP-3. The reaction took place at room temperature with gentle stirring and was stopped by adding 40 mM EDTA (330 μl of 0.5 M EDTA) after 24 hrs incubation.

SAMPLE PREPARATION FOR SEC ANALYSIS. The filters from the well-plates were soaked in THF to dissolve the rubber, and then the solute was filtered through Acrodisc 0.45 μm PTFE filters (Waters) to remove the gel fraction. The solute was then dried and prepared for SEC analysis. The rubber from the larger scale experiment was dissolved in freshly distilled THF and left in the dark in the refrigerator with periodic agitation until no visible gel was present. The solution was freeze-dried and the rubber was dissolved again in freshly distilled THF. Large-scale (WRP-3) samples were dialyzed using Nest Group's SpinDIALYZER (50 μL) with 0.6 μm polycarbonate membranes to remove the gel. The solution outside of the dialysis chamber and the gel within the chamber were freeze-dried until constant weight. Finally, the soluble fraction was dissolved in freshly distilled THF and filtered with 0.45 μm PTFE filters with sample concentrations between 0.5 and 0.8 mg/mL for SEC analysis.

SEC ANALYSIS. Molecular weights (MW)s and molecular weight distributions (MWD)s were determined by SEC using a Waters setup equipped with 6 Styragel columns (HR0.5, HR1, HR3, HR4, HR5, and HR6) thermostated at 35° C. as the stationary phase. THF freshly distilled from CaH2 was used as the mobile phase at a flow rate of 1 mL/min. The list of detectors include a Wyatt Technology Viscostar viscometer (VIS), a Wyatt Optilab DSP refractive index (RI) detector thermostated at 40° C., a Wyatt DAWN EOS 18 angle multiangle laser light scattering (MALLS) detector, a Wyatt quasi-elastic light scattering (QELS) and a Waters 2487 Dual Absorbance ultraviolet (UV) detector. The UV detector wavelength was set at 210 nm for polyisoprene in THF. Absolute molecular weights and radii of gyration were determined using ASTRA® V software 5.2.1.4 and dn/dc=0.130 reported for high cis-polyisoprene.30 Low MWs were estimated from the elution times using a calibration curve [log MW=11.06−0.13×elution time (min)] obtained with PIP standards, which agreed with that extrapolated from the LS data of the high MW rubber.

### Example I

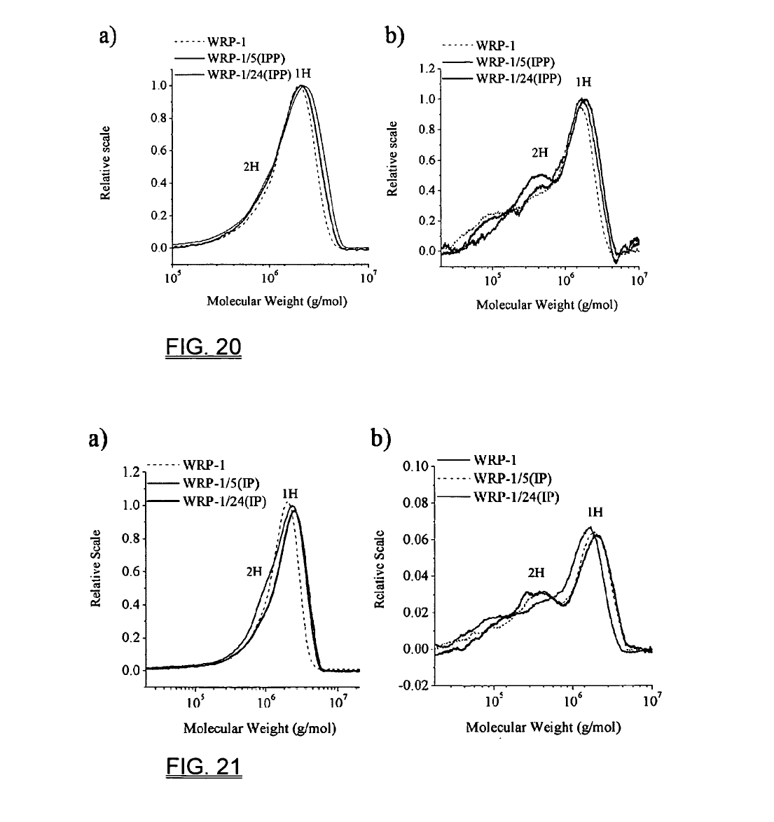

CONTROL EXPERIMENT. 2 μL of buffer (100 mM Tris-HCl, pH 7.5, 1.25 mM MgSO₄, 5 mM DTT), 0.4 μL (4×10⁻⁸ mol) of 100 mM IPP in water, 0.6 μL (6×10⁻⁸ mol) of 1 mM FPP in water and 2 mg of WRP-1 in 17 μL of water were added to microwell plates. The reactions were allowed to proceed for 5 hrs and 24 hrs at 25° C. in an incubator. The reactions were stopped by adding 40 μL (3.2×10⁻⁶ mol) of 80 mM EDTA in water. The filter plate was vacuumed and then washed two times with 150 μL water then once with 95% ethanol, then oven-dried at 37° C. for 30 minutes. The filters from the well-plates were immersed in THF to dissolve the rubber, and then the solute was filtered through Acrodisc 0.45 μm PTFE filters (Waters) to remove the gel fraction. The solute was then dried. The soluble fraction was dissolved in freshly distilled THF and filtered with 0.45 μm PTFE filters with sample concentrations between 0.5 and 0.8 mg/mL for SEC analysis. Table 2 summarizes the data. #1H and #2H refer to the SEC peaks in the high molecular weight region. (see FIG. 20)

TABLE 2
Summary of reaction conditions and SEC results for example I

| | | | | | SEC | | | | | |
|---|---|---|---|---|---|---|---|---|---|---|
| | | | | High MW | Elution | | Peak MW | | | |
| | | | Time | Fraction | time (min) | | g/mol × 10⁶ | | R_{g} (nm) | |
| Samples | IPP | FPP | (hrs) | ~wt % | #1H | #2H | #1H | #2H | #1H | #2H |
| WRP-1 | | | | 50 | 37.3 | — | 1.62 | — | 77.2 | — |
| WRP-1/5(IPP) | + | + | 5 | 70 | 37.3 | — | 1.62 | — | 78.1 | — |
| WRP-1/24(IPP) | + | + | 24 | 80 | 36.8 | 41.6 | 1.87 | 0.46 | 86.4 | 44.8 |

FIG. 20 compares the LS and RI traces of the WRP-1/5(IPP) and WRP-1/24(IPP) with WRP-1. WRP-1 had a main peak MW of ˜1.6×10⁶ g/mol. WRP-1 had a molecular weight tail in the high MW region from ˜2.28 to ˜7.41×10⁵ g/mol. The evidence of MW growth is supported by the increase of R_{g} and the increase of the high MW mass fraction. The 1H peak of WRP-1/24 shifted to higher molecular weight relative to WRP-1 and WRP-1/5, and a distinctive second peak (2H) appeared with an approximate molecular weight of ˜5×10⁵ g/mol. The approximate weight fraction of the high MW materials increased from ˜50% to ˜80%. This observation is similar to that reported in the literature by scintillation spectroscopy using radio-labeled ¹⁴C-IPP monomer; this peak was identified to be newly-formed NR. [72,116]

### Example II

2 μL of buffer (100 mM Tris-HCl, pH 7.5, 1.25 mM MgSO4, 5 mM DTT), 0.4 μL (4×10⁻⁸ mol) of 100 mM IP in ethanol, 0.6 μL (6×10⁻⁸ mol) of 1 mM FPP in water and 2 mg of WRP-1 in 17 μL of water. Reaction times of 5 hrs and 24 hrs were used at 25° C. in an incubator. The reactions were stopped by adding 40 μL (3.2×10⁻⁶ mol) of 80 mM EDTA in water. The filter plate was vacuumed and then washed two times with 150 μL water then once with 95% ethanol, then oven-dried at 37° C. for 30 minutes. Molecular weight characterization was performed as described above. The sample concentration ranged from 0.5 to 1.0 mg/mL. Table 3 summarizes the data. #1H and #2H refer to the SEC peaks in the high molecular weight region. (see FIG. 21)

TABLE 3
Summary of reaction conditions and SEC results for example II

| | | | | High | SEC | | | | | |
|---|---|---|---|---|---|---|---|---|---|---|
| | | | | MW | Elution | | Peak MW | | | |
| | | | Time | Fraction | time (min) | | g/mol × 10⁶ | | R_{g} (nm) | |
| Samples | IP | FPP | (hrs) | ~wt % | #1H | #2H | #1H | #2H | #1H | #2H |
| WRP-1 | | | | 50 | 37.3 | — | 1.62 | — | 77.2 | — |
| WRP-1/5(IP) | + | + | 5 | 80 | 36.7 | 42.2 | 1.93 | 0.38 | 89.8 | 41.4 |
| WRP-1/24(IP) | + | + | 24 | 80 | 36.6 | 41.8 | 1.99 | 0.43 | 98.5 | 44.0 |

FIG. 21 compares the LS and RI traces of the 5 and 24 hr samples using IP as monomer to replace IPP and plotted with WRP-1. The 1H peak of WRP-1/5(IP) and WRP-1/24(IP) shifted to higher molecular weight relative to WRP-1. The main peak of NR from WRP-1 grew to nearly 2.00×10⁶ g/mol; an indication of growth of natural rubber. A distinctive second peak (2H) appeared with an approximate molecular weight of ˜4×10⁵ g/mol, similar to the preceding example. This observation was similar to that reported in the literature by scintillation spectroscopy using radio-labeled ¹⁴C-IPP monomer; this peak was identified to be newly-formed NR. [72,116] The approximate weight fraction of the high MW materials increased to ˜80%, similar to that of examples that used IPP as monomer. When IP was used as monomer, similar results to conventional in vitro NR biosynthesis, where IPP is the monomer, were observed. Evidence of NR growth is supported by the increase of R_{g} and the increase of the high MW mass fraction.

### Example III

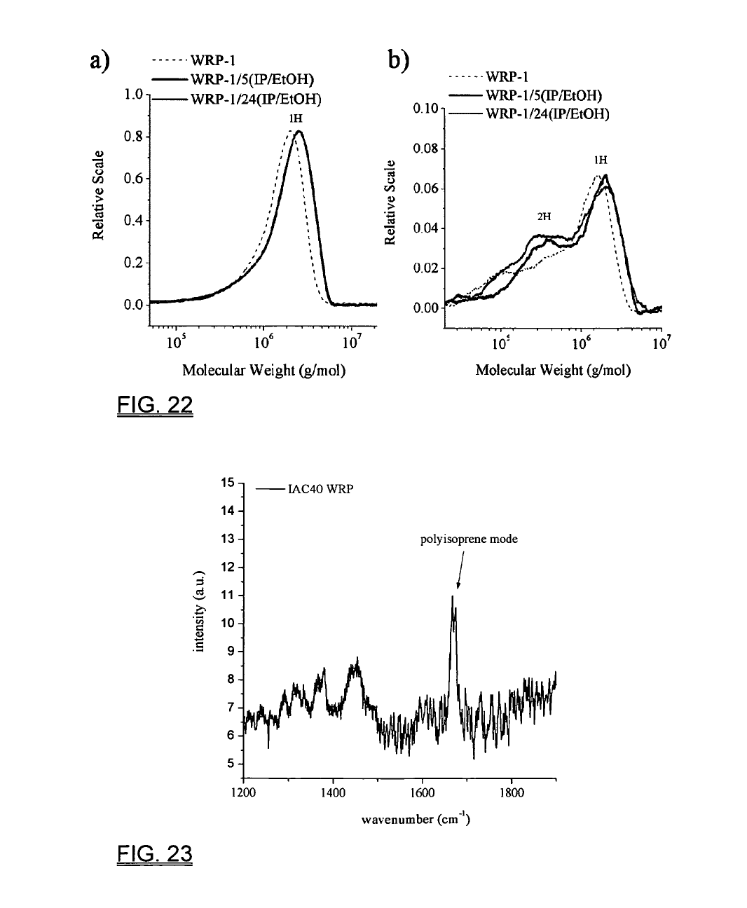

Utilizing the equipment and procedure like that of example I, 2 μL of buffer (100 mM Tris-HCl, pH 7.5, 1.25 mM MgSO₄, 5 mM DTT), 0.4 μL (4×10⁻⁸ mol) of 100 mM IP in ethanol, 0.6 μL (6×10⁻⁸ mol) of 1 mM FPP in water and 2 mg of WRP in 13 μL of water and 4 μL of 100% ethanol to a reaction volume of 20 μL were combined reaction times of 5 hrs and 24 hrs were used at 25° C. in an incubator. There were 9 wells for 5 hrs and 9 for 24 hrs to a total of 18 wells. The reactions were stopped by adding 40 μL (3.2×10⁻⁶ mol) of 80 mM EDTA in water. The filter plate was vacuumed and then washed two times with 150 μL water then once with 95% ethanol, then oven-dried at 37° C. for 30 minutes. Molecular weight characterization was followed like that of the preceding example. The sample concentration ranged from 0.5 to 1.0 mg/mL. Table 4 summarizes the data. #1H and #2H refer to the SEC peaks in the high molecular weight region. (see FIG. 22)

TABLE 4
Summary of reaction conditions and SEC results for example III

| | | | | | High | SEC | | | | | |
|---|---|---|---|---|---|---|---|---|---|---|---|
| | | | | | MW | Elution | | Peak MW | | | |
| | | | 10% | Time | Fraction | time (min) | | g/mol × 10⁶ | | R_{g} (nm) | |
| Samples | IP | FPP | EtOH | hrs | ~wt % | #1H | #2H | #1H | #2H | #1H | #2H |
| WRP-1 | | | | | 50 | 37.3 | — | 1.62 | — | 77.2 | — |
| WRP- | + | + | + | 5 | 90 | 36.5 | 42.2 | 2.05 | 0.38 | 99.8 | 41.4 |
| 1/5(IP/EtOH) | | | | | | | | | | | |
| WRP- | + | + | + | 24 | 90 | 36.6 | 42.1 | 1.99 | 0.38 | 98.5 | 41.4 |
| 1/25(IP/EtOH) | | | | | | | | | | | |

FIG. 22 compares the LS and RI traces of the 5 and 24 hr samples using IP as monomer in the presence of 10% (v/v) ethanol and plotted with WRP-1. The 1H peaks of WRP-1/5(IP/EtOH) and WRP-1/24(IP/EtOH) shifted to higher molecular weight relative to WRP-1, which are indications of growth of NR. From the SEC results, it was observed that WRP-1/5(IP/EtOH) and WRP-1/24(IP/EtOH) had similar molecular weights and increased the MW of NR from ˜1.62×10⁶ g/mol to nearly 2.00×10⁶ g/mol, a growth of ˜4×10⁵ g/mol. A distinctive second peak (2H) appeared with an approximate molecular weight of ˜4×10⁵ g/mol, similar to the preceding examples. This observation was similar to that reported in the literature by scintillation spectroscopy using radio-labeled ¹⁴C-IPP monomer; this peak was identified to be newly-formed NR. [72,116] The approximate weight fraction of the high MW materials increased to ˜90%; the NR isolated from the WRP after incubation increased by ˜80%. The evidence of MW growth is supported by the increase of R_{g} data and the increase of the high MW mass fraction.

### Example IV

In this example, a control experiment using RRIM600 WRP and IPP as the monomer was performed, at a scale that was enhanced to milliliter-scale to provide a facile method to analyze the mass gain of in vitro NR biosynthesis. In a 15 mL centrifuge tube, 76 mg of RRIM600 WRP-3 was suspended in 3705 μL of buffer (100 mM Tris-HCl, pH 7.5, 1.25 mM MgSO₄, 5 mM DTT). Subsequently, 38 μL (3.8×10⁻⁷ mol) of 10 mM IPP in water, 57 μL (5.7×10⁻⁸ mol) of 1 mM FPP in water was added to make up 3800 μL total reaction volume. The reaction took place at room temperature with gentle rotation and was stopped by adding 330 μL of 0.5M EDTA in water (final concentration of 43 mM) after 24 hours. The sample was then washed with excess buffer (100 mM Tris-HCl, pH 7.5, 1.25 mM MgSO₄, 5 mM DTT) and freeze-dried until constant weight.

Table 5 below presents the gravimetric analysis of WRP-3/24(IPP). The weight reported was determined after freeze-drying the samples in vacuum for over 5 days till constant weight was achieved. When compared to the initial WRP weight, a positive mass gain was observed, indicating that the IPP monomer was converted into polymer in vitro. The mass gain to the overall weight of WRP-3/24(IPP) after incubation was ˜46%.

TABLE 5
Gravimetric analysis of WRP-3/24(IPP)

| | Initial WRP | Wt. after | Mass balance |
|---|---|---|---|
| Sample | (mg) | incubation (mg) | (mg) |
| WRP-3/24(IPP) | 76 | 111 | +35 |

### Example V

Utilizing the equipment and procedure similar to example IV, in a 15 mL centrifuge tube, 76 mg of RRIM600 WRP-3 was suspended in 3705 μL of buffer (100 mM Tris-HCl, pH 7.5, 1.25 mM MgSO₄, 5 mM DTT). Subsequently, 38 μL (3.8×10⁻⁴ mol) of >99% IP (final concentration of 100 mM), 57 μL (5.7×10⁻⁸ mol) of 1 mM FPP in water was added to make up 3800 μL total reaction volume. The reaction took place at room temperature with gentle rotation and was stopped by adding 330 μL of 0.5M EDTA in water (final concentration of 43 mM) after 24 hours. The sample was then washed with excess buffer (100 mM Tris-HCl, pH 7.5, 1.25 mM MgSO₄, 5 mM DTT) and freeze-dried until constant weight was achieved.

Table 6 below presents the gravimetric analysis of WRP-3/24(IPP). The weight reported was determined after freeze-drying the samples in vacuum for over 5 days till constant weight was achieved. When compared to the initial WRP weight, a positive mass gain was observed, indicating that the IP monomer was converted into polymer in vitro. The mass gain to the overall weight of WRP-3/24(IP) after incubation was ˜35%.

TABLE 6
Gravimetric analysis of WRP-3/24(IP)

| | Initial WRP | Wt. after | Mass balance |
|---|---|---|---|
| Example | (mg) | incubation (mg) | (mg) |
| WRP-3/24(IP) | 76 | 103 | +27 |

In Example V, the amount of mass gain was approximately equal to that of isoprene monomer introduced into the system. 26 mg of isoprene monomer was introduced and 27 mg of mass gain was measured within experimental error. This suggests quantitative conversion of isoprene incorporation into natural rubber.

### Example VI

### Micro-Raman Monitoring of In Vitro NR Biosynthesis Using IP as Monomer

Materials

Isoprene (>99%) was purchased from Sigma-Aldrich. Isolation of enzymatic-active washed rubber particles from IAC40 Hevea was prepared as using similar methodology described in the literature [93,114].

Micro-Raman Spectroscopy

The measurement of characteristic vibration modes was performed using Horiba-JobinYvon LabRam Raman spectrophotometer equipped with x50 Mitutoyo long-working-distance objective, capable of penetrating the glass without a substantial glass fluorescence signal. The laser had the following setup: λ=647 nm, hole=300 μm, silt=150 μm, 300 seconds/spectrum, and software=Labspec 3.1. The samples were prepared by measuring 0.5 g of IAC40 WRP into 1 mL glass vials and then sealing the vials. The rubber content of IAC40 WRP is 21.18%. 0.15 mL (0.1022 g) of >99% isoprene (Sigma-Aldrich) was injected through the seal into the IAC40 WRP. The sample was subjected to agitation for one minute.

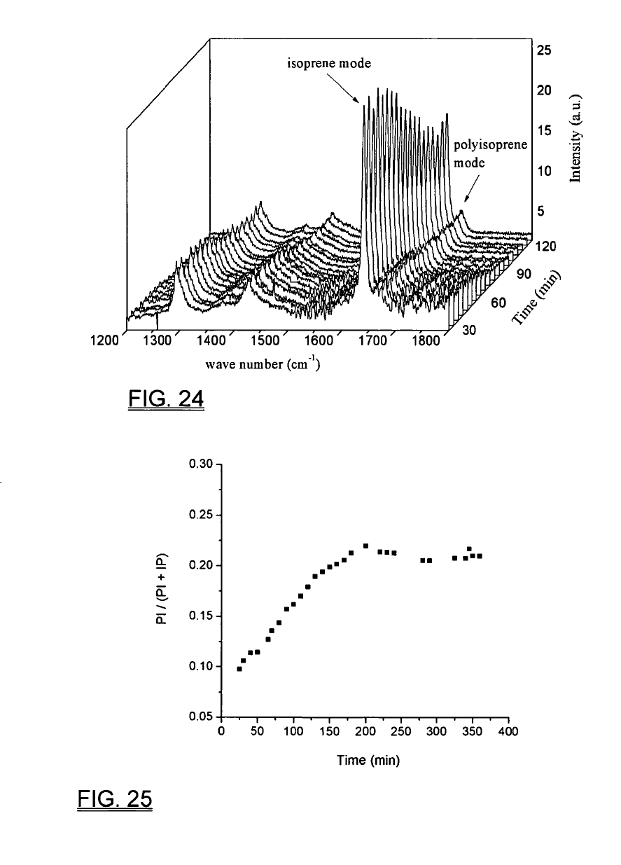

FIG. 23 shows a Raman spectrum of pure IAC40 WRP. The peak at 1670 cm⁻¹ is assigned as the polyisoprene vibration mode. The isoprene vibration mode is assigned at 1640 cm⁻¹.

FIG. 24 displays the Raman spectrum of IAC40 WRP after the addition of isoprene monomer from 30 to 120 minutes. The isoprene mode (1640 cm⁻¹) decreased over time as polyisoprene mode (1670 cm⁻¹) gradually increased. This suggests the conversion of isoprene into polyisoprene. We also noticed that upon addition of the isoprene, yellow spots appeared in the rubber particles, indicating ion formation.

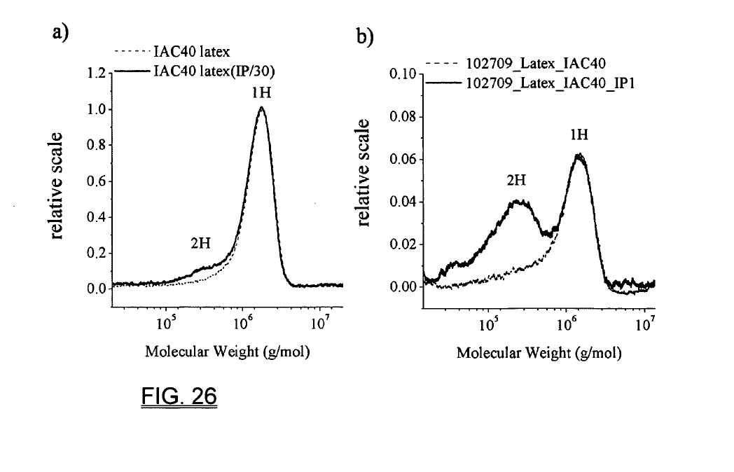

FIG. 25 presents the growth of polyisoprene mode for the duration of the Raman monitoring. The y-axis is a ratio of integrated polyisoprene peak and the total area of polyisoprene and isoprene. The peak areas were determined using a Lorentz fit protocol using Origin 8.0. From the Raman spectroscopy, the polyisoprene peak increased ˜12% over six hours. The enzymatic activity of IAC40 WRP lasted for approximately three hours.

### Example VII

Fresh latex was collected from Hevea Brasiliensis, immediately stabilized by a buffer solution (0.1M NaHCO₃ in water, 50% glycerol, 0.3% (w/v) NaN₃ in water, 5 mM cysteine in water), and shipped to the USDA (Albany, Calif.) at −80° C. The frozen latex was then shipped to the University of Akron at −80° C. and stored in a −80° C. freezer until use. The USDA determined the rubber content of the IAC40 latex to be 18.5 wt %. Before the experiment, frozen latex was allowed to thaw for 5 minutes to yield a liquid. 0.513 g of IAC40 latex was measured into a 1 mL glass vial and then the vial was sealed. 0.15 mL (0.1022 g) IP (>99%, Sigma-Aldrich) was injected through the seal into the IAC40 latex. The sample was shaken by hand for one minute and incubated at room temperature for 30 hours. After incubation, the sample was freeze-dried in the vacuum oven for a week until constant weight was achieved. For comparison purposes, another sample of pure IAC40 latex was prepared. Both samples were dissolved in freshly distilled THF and left in the dark in the refrigerator with periodic agitation until transparent. The NR solutions were dialyzed with 0.5 μm PTFE membranes to remove any micro-gel. The solution outside of the dialysis chamber was freeze-dried until constant weight. 0.9 mg of the incubated product was dissolved in 0.9478 mL of freshly distilled THF and filtered with 0.45 μm PTFE filters (VWR) to yield a 0.9495 mg/mL solution. 1.1 mg of isolated NR from pure IAC40 latex was dissolved in 1.4960 mL of freshly distilled THF and filtered with 0.45 μm PTFE filters (VWR) to yield a 0.7353 mg/mL solution. 100 μL of both solutions were injected onto the SEC columns for analysis. FIG. 26 displays the SEC traces and Table 7 summarizes the data. The main high molecular weight peak, designated as 1H peak, of the rubber obtained after IP incubation shifted slightly to higher molecular weight (1.94×10⁶ g/mol), compared to that of the control rubber isolated from the IAC40 latex (1.79×10⁶ g/mol), an indication of growth of natural rubber in the latex. A distinctive second peak (2H) appeared with an approximate molecular weight of ˜4×10⁶ g/mol. This is the largest new peak observed, in comparison with examples II and V, where WRP was incubated with IP. This also supported the formation of new rubber or the growth of existing shorter chains.

FIG. 26 shows SEC of IAC40 latex, and IAC40 latex (IP/30), High MW region. a) is LS trace, and b) is RI trace.

TABLE 7
Summary of reaction conditions and SEC results for example VII

| | | | High | SEC | | | | | |
|---|---|---|---|---|---|---|---|---|---|
| | | | MW | Elution | | Peak MW | | | |
| | | Time | Fraction | time (min) | | g/mol × 10⁶ | | R_{g} (nm) | |
| Samples | IP | (hrs) | ~wt % | #1H | #2H | #1H | #2H | #1H | #2H |
| IAC40 latex | | | 25 | 36.15 | — | 1.79 | — | 85.3 | — |
| IAC40 latex(IP/30) | + | 30 | 28 | 36.08 | 42.06 | 1.94 | 0.42 | 98.5 | 43.2 |

The approximate weight fraction of the high MW materials increased from 25% to 28%. Evidence of NR growth was supported by the increase of R_{g} and the increase of the high MW mass fraction. This increase inherently suggested that the IAC40 latex contained active enzymes and necessary components for the conversion of isoprene into polyisoprene without the conventional need to purify the latex into WRP.

Another aspect of the present invention is that by utilizing a different catalyst (in addition to the cis-prenyl transferase catalyst inherently contained in the natural rubber latex), a trans conformation can be made. For example, by using active enzyme from a species that produces trans-PIP (trans-prenyl transferase), trans-1,4-polyisoprene can be produced from isoprene. Using a mixture of cis- and trans-prenyltransferase a blend of cis- and trans-1,4-polyisoprene can be produced Depending upon the amount of the trans catalyst (for example, active particles from E. ulmoides, which produces trans rubber) added in comparison with the inherent cis-prenyl transferase catalyst, the ratio of the trans-1,4-polyisoprene to that of the cis-1,4-polyisoprene can be readily controlled.

The in vitro production of natural rubber from isoprene monomers can be utilized in any article wherein synthetic natural rubber is utilized. Numerous such end uses exists such as tires including passenger car tires, truck tires, off road tires, and the like; various types of conveyor belts; various types of drive belts such as utilized on engines and the like, medical gloves, and so forth.

Isoprenoids other than cis-1,4-polyisoprene or blends of cis-1,4-polyisoprene and trans-1,4-polyisoprene or solely trans-1,4-polyisoprene can be produced according to the present invention in a manner as noted hereinabove, hereby fully incorporated by reference, and include terpenes, carotenoids, fat soluble vitamins, ubiquinones, various steroids, various alkaloids, and the like. For example, in lieu of the cis-prenyl transferase cofactor enzyme, other enzymes of the transferase class can be utilized such as transaminases, transacetylases, transmethylases, and the like can be utilized to replicate themselves in the presence of the natural rubber latex derived from rubber trees utilizing appropriate initiators that effect carbocationic polymerization of isoprene to yield the desired isoprenoid. Examples of various terpenes include monocyclic terpenes such as dipentene, various dicyclic terpenes such as pinene, or various acyclic terpenes such as myrcene. Examples of terpene derivatives include camphor, menthol, terpineol, borneol, geraniol, and the like. Examples of fat soluble vitamins include vitamin A, D, E, and K. Examples of ubiquinones include Q or Q₁₀ also known as coenzyme Q or coenzyme Q₁₀. Examples of steroids include any of the lipids that contain a hydrogenated cyclopentanoperhydrophenanthrene ring system. Literally hundreds of compounds exist including progesterone, adrenocortical hormones, sex hormones, cardiac aglycones, bile acids, sterols such as cholesterol, saponins, some carcinogenic hydrocarbons, and squalene.

The reaction conditions for forming isoprenoids are similar to the conditions for forming the cis-1,4-polyisoprenes and the cis-1,4-polydienes and are hereby fully incorporated by reference such as the type of any amount of allylic initiators, the polymerization temperatures, molecular weight, and the like.

The above noted isoprenoids while produced by a different process route can be utilized for the current existing uses and applications known to the literature and to the art, including those set forth above such as vitamins, steroids, etc.

While in accordance with the patent statutes the best mode and preferred embodiment have been set forth, the scope of the invention is not intended to be limited thereto, but only by the scope of the attached claims.

What is claimed is:

1. A process for the synthetic production of cis-1,4-polyisoprene or cis-1,4-polydiene, comprising the steps of:
mixing, in any order, isoprene monomers or other diene monomers having from 4 to 8 carbon atoms, a natural rubber latex or washed rubber particles (WRP), and an allylic initiator, said latex or WRP containing an enzyme catalyst and a divalent cofactor therein; and
polymerizing said mixture and producing cis-1,4-polyisoprene or cis-1,4-polydiene.

2. The process of claim 1, wherein said allylic initiators comprise allylic pyrophosphates, including 1,1-dimethylallyl pyrophosphate, farnesyl pyrophosphate, geranyl-geranyl pyrophosphate, geranyl pyrophosphate, allylic neryl pyrophosphate, dimethyl allyl halide, dimethyl allyl bromide, dimethyl allyl chloride, dimethyl allyl iodide, dimethyl allyl fluoride, dimethyl allyl hydroxide, or a compound containing an allylic pyrophosphate end group, or a functionalized allylic pyrophosphate; wherein said polymerization occurs at a temperature of from about 10° C. to about 60° C.; and wherein the number of cis-1,4-isoprene repeat units or other cis-1,4-diene repeat units in said polymer is from about 5 to about 30,000.

3. The process of claim 2, wherein said monomers are said isoprene monomers or 1,3-butadiene or 2,3-dimethyl-butadiene-1,3 monomers, including washing said natural rubber latex prior to combining it with the isoprene or said other diene monomers, and wherein said enzyme comprises cis-prenyl transferase, and wherein said cofactor comprises Mg⁺² or Mn⁺².

4. The process of claim 3, wherein said polymerization is a living polymerization.

5. The process of claim 4, wherein said allylic initiators comprise 1,1-dimethylallyl pyrophosphate, farnesyl pyrophosphate, geranyl-geranyl pyrophosphate, geranyl pyrophosphate, and allylic neryl pyrophosphate.

6. The process of claim 5, wherein said polymer is cis-1,4-polyisoprene

7. A process for the in vitro production of natural rubber comprising the steps of:
adding an allylic initiator and isoprene monomers or one or more other diene monomers having from 4 to 8 carbon atoms to a natural rubber latex or washed rubber particles containing an enzyme catalyst and optionally a divalent cofactor; and
carbocationically or electrophilically polymerizing said isoprene or said one or more other diene monomers, optionally in the presence of pyrophosphoric acid or salts thereof, to produce cis-1,4-polyisoprene or cis-1,4-polydiene.

8. The process of claim 7, wherein said allylic initiators comprise allylic pyrophosphates including 1,1-dimethylallyl pyrophosphate, farnesyl pyrophosphate, geranyl-geranyl pyrophosphate, geranyl pyrophosphate, allylic neryl pyrophosphate, dimethyl allyl halide, dimethyl allyl bromide, dimethyl allyl chloride, dimethyl allyl iodide, dimethyl allyl fluoride, dimethyl allyl hydroxide, or a compound containing an allylic pyrophosphate end group, or a functionalized allylic pyrophosphate, and wherein the number of cis-1,4-isoprene or cis-1,4-diene repeat units in said polymer is from about 5 to about 30,000.

9. The process of claim 8, including washing said natural rubber latex prior to combining it with said isoprene monomers or said other diene monomers, and wherein said enzyme comprises cis-prenyl transferase.

10. The process of claim 9, including said cofactor, said cofactor comprising Mg⁺² or Mn⁺²; wherein said polymerization occurs at a temperature of from about 10° C. to about 60° C.; and wherein the number of cis-1,4-polyisopene or cis-1,4-diene repeat units in said polymer is from about 10 to about 25,000.

11. The process of claim 10, wherein said allylic initiators comprise 1,1-dimethylallyl pyrophosphate, farnesyl pyrophosphate, geranyl-geranyl pyrophosphate, geranyl pyrophosphate, and allylic neryl pyrophosphate.

12. The process of claim 9, wherein said polymerization is a living polymerization.

13. The process of claim 10, wherein said polymer is cis-1,4-polyisoprene.

14. A natural rubber composition comprising:
polymers of cis-1,4-polydienes other than cis-1,4-polyiosprene that are essentially free of non-enzyme catalysts therein.

15. The natural rubber according to claim 14, wherein said polymers contain from about 5 to about 30,000 repeat units of cis-1,4-diene; and wherein said natural rubber composition generally contains less than about 10 ppm of an non-enzyme for every 100 parts by weight of said polymer.

16. The natural rubber composition of claim 15, wherein said cis-1,4-polydiene is derived from an allylic initiator, an enzyme catalyst, and a divalent metal cofactor.

17. A process for the in vitro production of isoprenoids, comprising the steps of:
forming a solution comprising an allylic pyrophosphate initiator, isoprene or other diene monomers containing from 4 to 8 carbon atoms, an enzyme catalyst and optionally a cofactor; and
carbocationically polymerizing said isoprene and said other diene monomers to produce a polyisoprenoid composition.

18. The process of claim 17, wherein said polyisoprenoid comprises a terpene, a carotenoid, a fat soluble vitamin, a ubiquinone, a steroid, or an alkaloid.

19. The process of claim 18, including said cofactor, wherein said allylic initiators comprise allylic pyrophosphates, including 1,1-dimethylallyl pyrophosphate, farnesyl pyrophosphate, geranyl-geranyl pyrophosphate, geranyl pyrophosphate, allylic neryl pyrophosphate, dimethyl allyl halide, dimethyl allyl bromide, dimethyl allyl chloride, dimethyl allyl iodides, dimethyl allyl fluoride, dimethyl allyl hydroxide, or a compound containing an allylic pyrophosphate end group, or a functionalized allylic pyrophosphate.

20. The process of claim 19, wherein the polymerization temperature is from about 10° C. to about 60° C.
